(19) United States
(12) Patent Application Publication
(10) Pub. No.: US 2023/0258545 A1
(43) Pub. Date: Aug. 17, 2023

(54) SAMPLE PREPARATION METHOD, CELL ANALYSIS METHOD, SAMPLE PREPARATION APPARATUS, AND CELL ANALYZER

(71) Applicant: SYSMEX CORPORATION, Kobe-shi (JP)

(72) Inventors: Hiroaki INOKUCHI, Kobe-shi (JP); Toshikuni SUGANUMA, Kobe-shi (JP); Kei SHIRASUNA, Kobe-shi (JP)

(73) Assignee: SYSMEX CORPORATION, Kobe-shi (JP)

(21) Appl. No.: 18/309,161

(22) Filed: Apr. 28, 2023

Related U.S. Application Data
(63) Continuation of application No. PCT/JP2021/038129, filed on Oct. 14, 2021.

(30) Foreign Application Priority Data
Oct. 29, 2020 (JP) 2020-181121

(51) Int. Cl. G01N 1/38; G01N 33/483

(52) U.S. Cl. CPC G01N 1/38; G01N 33/4833; G01N 35/1009

(57) ABSTRACT

Automation of analysis of cells contained in different types of specimens collected from different organs or sites is enabled. This sample preparation method is a method for preparing a sample for analyzing cells contained in a specimen, and includes: obtaining a dispersion condition corresponding to the type of the specimen; and dispersing the cells aggregated and contained in the specimen, according to the obtained dispersion condition.

### CROSS REFERENCE TO RELATED APPLICATIONS

This application is a continuation of International Application PCT/JP2021/038129 filed on Oct. 14, 2021, which claims benefit of Japanese patent application JP 2020-181121 filed on Oct. 29, 2020, both of which are incorporated herein by reference in their entireties.

### BACKGROUND OF THE INVENTION

### Field of the Invention

The present invention relates to sample preparation for analyzing cells contained in a specimen.

### Description of the Background Art

Japanese Laid-Open Patent Publication No. 2015-96847 discloses a canceration information providing apparatus that provides canceration information of cervical cancer on the basis of the size of a cell (C), the size of the cell nucleus (N) of the cell, and the DNA content of the cell regarding each cell contained in a sample collected from epithelial cells of the uterine cervix. In this apparatus, collected aggregated cells are subjected to pretreatment such as dispersion, DNA staining, and the like to prepare a measurement sample, and the size of a cell (C), the size of the cell nucleus (N) of the cell, and the DNA content of the cell regarding each cell in the measurement sample are measured by a flow cytometer, whereby canceration information of cells is provided. Such an automated apparatus can contribute to speed-up of cancer diagnosis.

Automation of cancer diagnosis is desired not only for cervical cancer but also for other types of cancers. For example, for gynecological items, if cytology of cervical cancer and endometrial cancer can be performed by a single automated apparatus, improvement of efficiency of tests can be expected.

With respect to the apparatus of Japanese Laid-Open Patent Publication No. 2015-96847, it is disclosed that not only a sample collected from epithelial cells of the uterine cervix, but also epithelial cells of the oral cavity, the bladder, the pharynx, and organs can be the target of the analysis. Cells contained in different analysis targets have sizes and shapes different from those of a sample collected from epithelial cells of the uterine cervix. Therefore, in order to perform highly accurate analysis, it is necessary to manually perform pretreatments according to the respective samples. That is, in the apparatus of Japanese Laid-Open Patent Publication No. 2015-96847, the analysis work has not been able to be automated for different types of samples.

### SUMMARY OF THE INVENTION

The present inventors conducted thorough studies, and found that, when a dispersion condition for cells is made different in accordance with the type of a specimen, a useful analysis result can be obtained. On the basis of this finding, a sample preparation method according to a first aspect of the invention includes: obtaining a dispersion condition corresponding to a type of the specimen; and dispersing the cells aggregated and contained in the specimen, according to the obtained dispersion condition.

A cell analysis method according to a second aspect of the invention is a cell analysis method for analyzing proliferative capacity of each cell contained in a specimen, and includes: preparing a sample by the sample preparation method according to the first aspect of the invention; staining the cell; detecting optical information from the cell in the sample; and analyzing a measurement item regarding the proliferative capacity of the cell, on the basis of the detected optical information.

A sample preparation apparatus according to a third aspect of the invention is a sample preparation apparatus configured to prepare a sample for analyzing cells contained in a specimen, and includes: a dispersion processing part configured to disperse the cells aggregated and contained in the specimen; and a controller programmed to obtain a dispersion condition corresponding to a type of the specimen and control the dispersion processing part so as to perform a dispersion process on the specimen according to the obtained dispersion condition

A cell analyzer according to a fourth aspect of the invention is a cell analyzer configured to analyze proliferative capacity of each cell contained in a specimen, and includes: a preparation part implemented as the sample preparation apparatus according to the third aspect of the invention; a detection part configured to detect optical information from the cell in the sample prepared by the preparation part; and an analysis part configured to analyze a measurement item regarding the proliferative capacity of the cell, on the basis of the optical information obtained by the detection part.

### BRIEF DESCRIPTION OF THE DRAWINGS

FIG. 1 is a schematic plan view showing a configuration example of a sample analyzer;

FIG. 2 is a block diagram of a measurement apparatus;

FIG. 3 is a schematic diagram for describing an example of a first dispersion processing part;

FIG. 4 is a schematic diagram for describing an example of a discrimination and replacement part;

FIG. 5 is a schematic diagram for describing an example of a second dispersion processing part;

FIG. 6 is a schematic diagram for describing an example of a detection part;

FIG. 7 is a graph for describing morphological information of a cell obtained from optical information;

FIG. 8 is a block diagram showing a configuration of a computer usable as an analysis part;

FIG. 9 is a diagram showing an example of a plurality of dispersion conditions;

FIG. 10 is a schematic diagram for describing cells contained in a specimen of the uterine cervix and cells contained in a specimen of the uterine body;

FIG. 11 is a flowchart showing a specific example of a step of obtaining a dispersion condition corresponding to a specimen;

FIG. 12 is a diagram for describing a process of obtaining specimen information;

FIG. 13 is a flowchart for describing an analysis operation of a cell analyzer;

FIG. 14 is a flowchart for describing a sample preparation process in FIG. 13;

FIG. 15 is a flowchart for describing a first dispersion process in FIG. 14;

FIG. 16 is a flowchart for describing a second dispersion process in FIG. 14;

FIG. 17 is a graph showing comparison between detection results for respective dispersion conditions according to Example 1;

FIG. 18 is a graph showing comparison between detection results for respective dispersion conditions according to Example 2;

FIG. 19 is a flowchart showing a process of selecting a method for obtaining a dispersion condition, according to a modification;

FIG. 20 is a diagram showing an example of a selection screen for receiving a selection of a dispersion condition; and

FIG. 21 is a diagram showing an example of an input screen for receiving an input of a dispersion condition.

### DESCRIPTION OF THE PREFERRED EMBODIMENTS

[Outline of Sample Preparation Method]

An outline of the sample preparation method of the present embodiment will be described.

The sample preparation method is a method for preparing a sample for analyzing cells contained in a specimen. The sample preparation method is a method performed by a sample preparation apparatus that prepares a sample. The sample prepared by the sample preparation method is subjected to analysis performed by a cell analyzer. Through analysis of cells contained in the specimen, information regarding a disease of a subject can be obtained.

A specimen is a biological sample collected from a subject. The specimen contains cells of the subject. The subject is mainly a human, but may be an animal other than a human. The specimen contains cells derived from an organ and/or site of the subject. Preferably, the specimen is a biological sample collected from a site selected from the uterine cervix, the uterine body, the oral cavity, the esophagus, and the bronchus. The specimen need not be as in the state when the specimen has been collected. For example, when the specimen has been collected from a part of a tissue of a subject, the specimen may be in a form in which the collected substance is suspended in a preservation liquid. The sample preparation method is a method for processing the specimen into a sample suitable for being subjected to an analysis process performed by an analyzer.

In a specimen, cells are not necessarily present in a state where cells are individually separated from each other, and may be present in a form of a population where a plurality of cells are aggregated. In cell analysis, in order to suppress variation in the detection result of cells, it is important to separate (disperse) each aggregated cell population into single cells. The structure and the form (size and shape) of a cell are different depending on the organ and/or site from which the cell is derived. Therefore, the degree of aggregation of the cell population and the ease of separation thereof are different depending on the type of the specimen. Therefore, it is important to perform an appropriate cell dispersion process for each specimen for which the degree of aggregation and the ease of separation are different. The above dispersion means a process of separating each aggregated cell population contained in a specimen into single cells, thereby increasing the number of single cells contained in the specimen.

The sample preparation method of the present embodiment includes at least: a step of obtaining a dispersion condition corresponding to the type of a specimen; and a step of dispersing aggregated cells contained in the specimen, according to the obtained dispersion condition.

[Outline of Cell Analysis Method]

An outline of the cell analysis method of the present embodiment will be described.

The cell analysis method is a method for analyzing the proliferative capacity of each cell contained in a specimen. The cell analysis method is a method in which a sample is prepared by the sample preparation method and cells contained in the prepared sample are detected and analyzed. The cell analysis method is performed by a cell analyzer that performs cell analysis of a sample.

The cell analysis method includes at least: a step of preparing a sample by the sample preparation method of the present embodiment; a step of detecting optical information from each cell in the sample; and a step of analyzing a measurement item regarding the proliferative capacity of the cell, on the basis of the detected optical information.

[Cell Analyzer]

A cell analyzer 300 of the present embodiment will be described. As a configuration example of the sample preparation apparatus of the present embodiment, a configuration of a preparation part 110 of the cell analyzer 300 will also be described. In the following, an example in which the sample preparation apparatus is configured as the preparation part 110 of the cell analyzer 300 is shown. However, the sample preparation apparatus may be configured as a single apparatus.

The cell analyzer 300 prepares a sample for measurement from a specimen collected from a patient, detects each cell in the sample by means of a flow cytometer, and analyzes detected optical information (forward scattered light, side scattered light, side fluorescence), thereby performing analysis regarding whether or not a cancer cell is contained in the specimen.

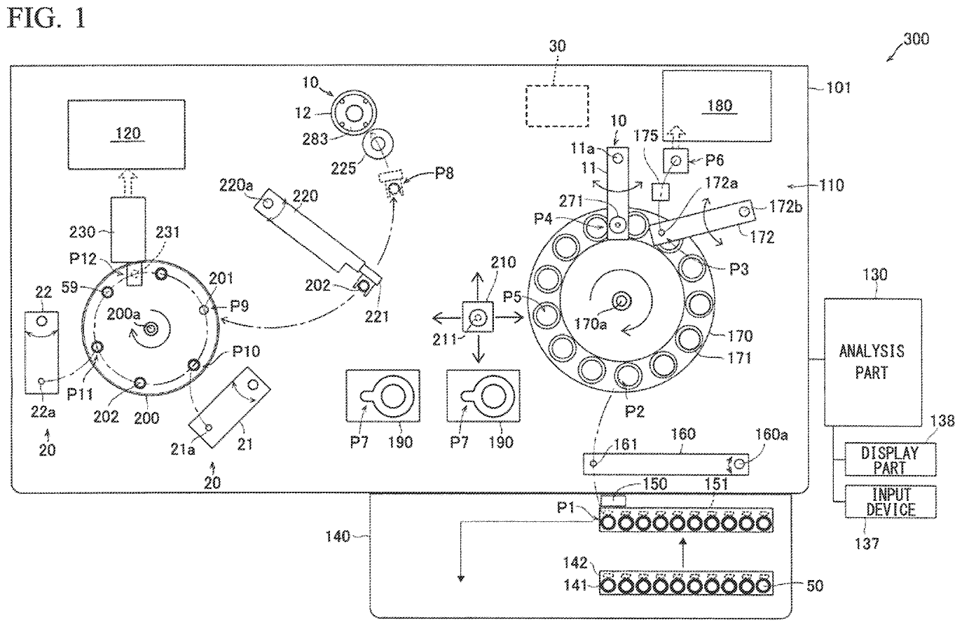

As shown in FIG. 1, the cell analyzer 300 includes a measurement apparatus 101 which prepares a sample 59 from a specimen 50 collected from a subject, and performs optical measurement on the sample 59. The measurement apparatus 101 includes: the preparation part 110; and a detection part 120 which detects optical information from each cell in the sample prepared by the preparation part 110. The cell analyzer 300 includes: an analysis part 130 communicably connected to the measurement apparatus 101.

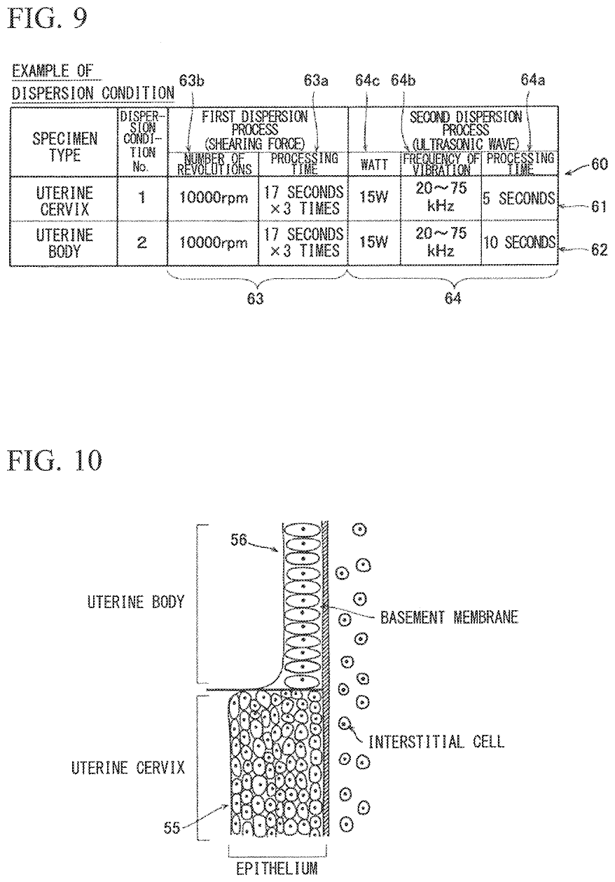

The preparation part 110 is implemented as a sample preparation apparatus including: a dispersion processing part 10 which disperses aggregated cells contained in the specimen 50; a staining liquid addition part 20 which adds, to the specimen 50, a staining liquid that stains cells; and a controller 30 which obtains a dispersion condition 60 (see FIG. 9) corresponding to the type of the specimen 50 and controls the dispersion processing part 10 so as to perform a dispersion process on the specimen 50 according to the obtained dispersion condition 60. In the example in FIG. 1, the dispersion processing part 10 includes a first dispersion processing part 11 and a second dispersion processing part 12. The staining liquid addition part 20 includes a first reagent addition part 21 and a second reagent addition part 22.

The preparation part 110 further includes a specimen setting part 140, an information reading part 150, a specimen dispensing part 160, a reaction table 170, a cell number measurement part 180, a discrimination and replacement part 190, and a reaction part 200. The preparation part 110 further includes a sample dispensing part 210, a container transfer part 220, and a sample suction part 230.

The specimen setting part 140 receives a plurality of specimen containers 141 each storing a specimen 50. The specimen container 141 stores a mixture of a preservation liquid having methanol as a main component, and a specimen 50. The plurality of specimen containers 141 are held by a specimen rack 142, in an upright state with their openings oriented upward. The specimen setting part 140 includes a conveyance belt so as to sequentially transport each specimen container 141 to a specimen suction position P1 for a specimen dispensing part 160, by moving a specimen rack 142 placed on a setting face.

The information reading part 150 is configured to read information in an information storage medium 151 attached to each specimen container 141. The information reading part 150 includes a bar code scanner or a camera. The information storage medium 151 is a bar code (one-dimensional code), a two-dimensional code, or the like, for example. The information storage medium 151 stores specimen information therein. The specimen information at least includes a specimen ID. The specimen ID is unique individual identification information for identifying an individual specimen 50, and is a combination of alphanumeric characters, for example. The information reading part 150 reads the specimen ID, as specimen information 90, from the information storage medium 151, and transmits the specimen ID to the controller 30. The information reading part 150 may be an RFID reader that reads later-described specimen information from the information storage medium 151 implemented as an RFID tag.

The specimen dispensing part 160 has a rotation shaft 160a at one end, and a pipette 161 which suctions and discharges the specimen 50, at the other end. The specimen dispensing part 160 is configured to rotate about the rotation shaft 160a, thereby being able to move the pipette 161 between a position above a specimen container 141 at the specimen suction position P1 and a position above a container 171 at a specimen discharge position P2. The specimen dispensing part 160 suctions the specimen 50 in the specimen container 141 and, at the specimen discharge position P2, dispenses the specimen 50 into a container 171 held by the reaction table 170.

The reaction table 170 is a round rotary table rotatable about a rotation shaft 170a and holds a plurality of containers 171 so as to be arranged along the circumferential direction. The reaction table 170 sequentially transfers, through rotation, the plurality of containers 171 to the specimen discharge position P2, a reagent dispensing position P3, and a first dispersion processing position P4.

Each container 171 has a tubular shape of which the upper part is open and of which the bottom is closed, and can store a specimen 50 therein. The container 171 can receive, through the upper opening, the pipette 161, a rotor 271 of the first dispersion processing part 11 described later, or the like.

A reagent dispensing part 172 includes a pipette 172a at one end and a rotation shaft 172b at the other end. The pipette 172a is connected, through a channel, to a reagent container set inside the measurement apparatus 101. The reagent dispensing part 172 is rotatable about the rotation shaft 172b, and can move the pipette 172a to the reagent dispensing position P3 and a dispensing position P6 for the cell number measurement part 180. The reagent dispensing part 172 dispenses a predetermined amount of a reagent sent from a reagent container, into a container 171. The reagent dispensing part 172 dispenses a diluent, as a reagent, into a container 171 having a specimen 50 dispensed therein. The reagent dispensing part 172 dispenses a lubricant reagent, as a reagent, into a container 171 having been subjected to a first dispersion process. The lubricant reagent contains a surfactant, and inhibits nonspecific attachment of cells 51 to the inner wall of the container 171. The reagent dispensing part 172 suctions the specimen 50 in the container 171 and dispenses the suctioned specimen 50 at the dispensing position P6. The specimen 50 dispensed at the dispensing position P6 is supplied, through a channel, to the cell number measurement part 180. Every time the reagent dispensing part 172 suctions a specimen 50, the reagent dispensing part 172 repeats suction and discharge of a washing liquid in a washing mechanism 175, thereby washing the pipette 172a.

The first dispersion processing part 11 includes a rotation shaft 11a at one end and the rotor 271 at the other end, and is configured such that the rotor 271 can move between the first dispersion processing position P4 and a withdrawal position that is shifted from the position above the reaction table 170. The first dispersion processing part 11 is also configured to be movable in the up-down direction by a mechanism described later, and is capable of downwardly moving the rotor 271 described later into a container 171 at the first dispersion processing position P4 and upwardly moving the rotor 271 from inside the container 171 to an upper outer position. The first dispersion processing part 11 is configured to perform a first dispersion process of mechanically applying a shearing force to aggregated cells in the specimen 50. The configuration of the first dispersion processing part 11 will be described later in detail.

The sample dispensing part 210 is configured to suction the specimen 50 in a container 171, and to dispense the suctioned specimen 50 into components of the preparation part 110. The sample dispensing part 210 includes a pipette 211, is held so as to be suspended from a position above and within the measurement apparatus 101 via an X-Y movement mechanism, and is configured to be able to move the pipette 211 in the up-down direction and the horizontal direction. The sample dispensing part 210 can move the pipette 211 to above each of a sample suction position P5 in the reaction table 170, a dispensing position P7 in the discrimination and replacement part 190, and a dispensing position P8 for a second container 202.

The cell number measurement part 180 is implemented as a flow cytometer that measures the number of measurement target cells contained in a specimen 50. The cell number measurement part 180 measures the number of cells in the specimen 50 on which the first dispersion process has been performed. In other words, the cell number measurement part 180 measures the number of aggregated cell populations in the specimen 50 in a stage before a second dispersion process is performed by the second dispersion processing part 12. In accordance with the measured number of cell populations, a dispensing condition (dispensing amount) is obtained.

The sample dispensing part 210 supplies the suctioned specimen 50 to the discrimination and replacement part 190 at the dispensing position P7 in the discrimination and replacement part 190. The discrimination and replacement part 190 separates cells 51 (see FIG. 4) from the specimen 50 processed by the first dispersion processing part 11, and subjects a specimen containing the separated cells 51 to the second dispersion processing part 12. The discrimination and replacement part 190 discriminates the measurement target cells (the cells 51) 50 contained in the specimen from the other cells (red blood cells, white blood cells, bacteria, etc.). The discrimination and replacement part 190 has a function of receiving the specimen 50 having been subjected to the first dispersion process performed by the first dispersion processing part 11 and transferring the specimen in the preservation liquid having methanol as a main component, into a replacement container 191 (see FIG. 4), to dilute the specimen by a diluent, thereby replacing the main component from methanol to the diluent. The discrimination and replacement part 190 condenses the measurement target cells contained in the specimen 50 in order to cause a prepared sample 59 to contain a necessary number of cells. In the example in FIG. 1, two discrimination and replacement parts 190 are provided.

The sample dispensing part 210 suctions the specimen 50 having been processed by the discrimination and replacement part 190, and discharges the suctioned specimen 50 into a second container 202 at the dispensing position P8 for the second container 202.

The second container 202 is set in advance at the reaction part 200. The second container 202 is transferred by the container transfer part 220, from the reaction part 200 to the dispensing position P8 for the second container 202.

The container transfer part 220 grips the second container 202 by means of a grip part 221, and transfers the second container 202 to the dispensing position P8 for the second container 202, the second dispersion processing part 12, the liquid removal part 225, and the reaction part 200. The container transfer part 220 is configured to move the grip part 221 along a circumferential locus about a rotation shaft 220a. The container transfer part 220 can move the grip part 221 in the up-down direction. The container transfer part 220 is configured to transfer, to the second dispersion processing part 12, the second container 202 having received the specimen 50 at the dispensing position P8 for the second container 202.

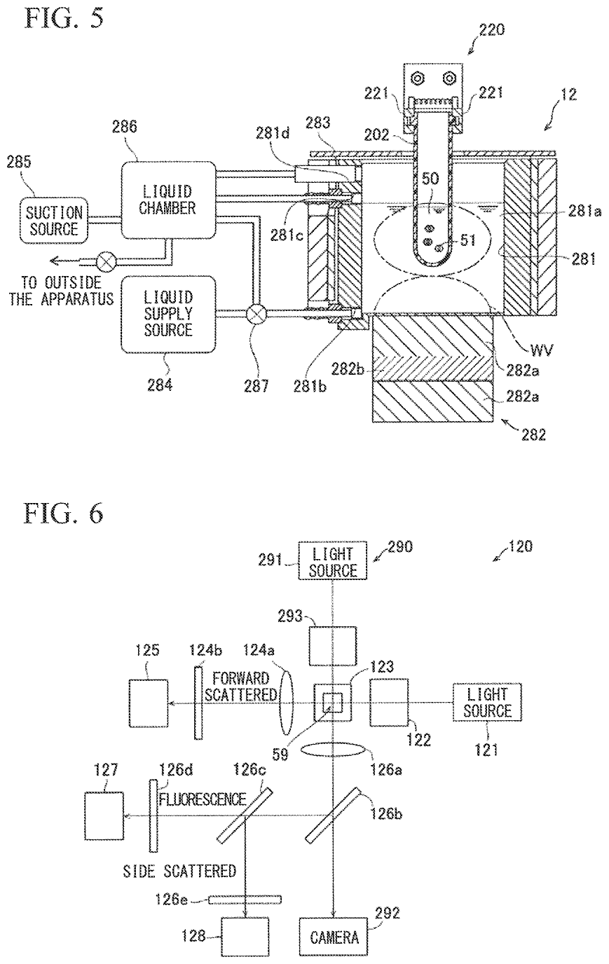

The second dispersion processing part 12 is configured to disperse aggregated cells 51 by applying ultrasonic vibration to the specimen 50. The second dispersion processing part 12 performs the second dispersion process on the specimen 50 for which the first dispersion process has been executed by the first dispersion processing part 11 and in which the concentration of the measurement target cells has been adjusted in the discrimination and replacement part 190. Accordingly, the second dispersion processing part 12 disperses aggregated cells remaining after the first dispersion process into single cells. The second dispersion process is executed in a state (see FIG. 5) where the second container 202 held by the container transfer part 220 is immersed in a liquid, as described later. The configuration of the second dispersion processing part 12 will be described later in detail.

The container transfer part 220 is configured to transfer, to the liquid removal part 225, the second container 202 having been subjected to the second dispersion process performed by the second dispersion processing part 12.

The liquid removal part 225 removes (drains) the liquid attached to the outer surface of the second container 202, after the second dispersion process performed by the second dispersion processing part 12. The liquid removal part 225 is configured to remove droplets attached to the outer surface of the second container 202 by supplying an airflow to the outer surface of the second container 202.

The container transfer part 220 is configured to transfer, to the reaction part 200, the second container 202 having been subjected to the liquid removal performed by the liquid removal part 225.

The reaction part 200 is implemented as a round rotary table rotatable about a rotation shaft 200a. A plurality of holders 201 each capable of having a second container 202 set therein are provided in an outer peripheral portion of the reaction part 200. The second container 202 is set in this holder 201 by the user. At a delivery position P9 on the path of the holder 201 according to rotation of the reaction part 200, the container transfer part 220 can take out the second container 202 from the holder 201, and can set the second container 202 to the holder 201.

The second container 202 has a tubular shape of which the upper part is open and of which the bottom is closed, and can store a liquid therein. The second container 202 is a small container having a volume smaller than that of the container 171.

The reaction part 200 is configured to transfer the second container 202 set in the holder 201 to a first reagent addition position P10 for the first reagent addition part 21, and to a second reagent addition position P11 for the second reagent addition part 22. At each addition position, a reagent (staining liquid) for a staining process is added. The reaction part 200 heats the second container 202 set in the holder 201 to a predetermined temperature, to accelerate the reaction between the specimen 50 and the reagent. Thus, the reaction part 200 is configured to cause the specimen 50 on which the dispersion process has been performed by the second dispersion processing part 12 and the staining liquid added by the staining liquid addition part 20, to react with each other.

As described above, the preparation part 110 (sample preparation apparatus) includes: the discrimination and replacement part 190 which separates the cells 51 from the specimen 50 processed by the first dispersion processing part 11 and subjects the separated cells 51 to the second dispersion processing part 12; and the reaction part 200 which causes the specimen 50 on which the dispersion process has been performed by the second dispersion processing part 12 and the staining liquid added by the staining liquid addition part 20, to react with each other. With this configuration, an ultrasonic dispersion process can be performed by the second dispersion processing part 12 on the specimen 50 having been subjected to the mechanical dispersion process of applying a shearing force by the first dispersion processing part 11. Therefore, a more effective dispersion process can be performed. Between the first dispersion process and the second dispersion process, the cells 51 can be separated from other unnecessary substances and condensed by the discrimination and replacement part 190. The staining liquid is added to the specimen 50 having been subjected to these processes, thereby causing the specimen 50 to react in the reaction part 200, whereby a sample suitable for cell analysis can be prepared.

The first reagent addition part 21 and the second reagent addition part 22 each supply a reagent for a staining process (staining liquid) into the second container 202 set in a holder 201 of the reaction part 200. The first reagent addition part 21 and the second reagent addition part 22 are set at positions in the vicinity of the peripheral edge of the reaction part 200. The first reagent addition part 21 has a supply portion 21a movable to the first reagent addition position P10 above the second container 202 set in the reaction part 200. Similarly, the second reagent addition part 22 has a supply portion 22a movable to the second reagent addition position P11. Accordingly, when the second container 202 has been transported to each of the first reagent addition position P10 and the second reagent addition position P11 by the reaction part 200, a predetermined amount of a reagent is added into the second container 202.

The reagent that is added by the first reagent addition part 21 is Rnase for performing RNA processing on cells. The reagent that is added by the second reagent addition part 22 is a staining liquid for performing PI staining. The RNA processing is a process for degrading RNA in cells. The PI staining liquid is a fluorescent staining liquid containing propidium iodide (PI). Since PI selectively stains the nucleus in each cell, fluorescence from the nucleus becomes able to be detected. After the first dispersion process and the second dispersion process have been executed, the staining liquid (the Rnase and the PI staining liquid) are added.

Upon addition of the staining liquid, sample preparation by the preparation part 110 is completed.

The sample suction part 230 has a function of suctioning the sample 59 in the second container 202 set in the reaction part 200, and transferring the sample 59 to the detection part 120. The sample suction part 230 is set at a position in the vicinity of the peripheral edge of the reaction part 200, and has a pipette 231 movable to a suction position P12 for the second container 202 set in the reaction part 200. The sample suction part 230 suctions the sample 59 from the inside of the second container 202 transported to the suction position P12 by the reaction part 200. The sample suction part 230 is connected, through a channel, to a later-described flow cell 123 (see FIG. 6) of the detection part 120. The sample suction part 230 supplies the sample 59 suctioned by the pipette 231, to the flow cell 123 shown in FIG. 6 of the detection part 120.

The detection part 120 detects optical information from each cell 51 in the sample 59 prepared by the preparation part 110. The detection part 120 is implemented as a flow cytometer shown in FIG. 6.

The analysis part 130 is configured to analyze a measurement item regarding the proliferative capacity of each cell 51, on the basis of the optical information obtained by the detection part 120.

<Details of Measurement Apparatus 101>

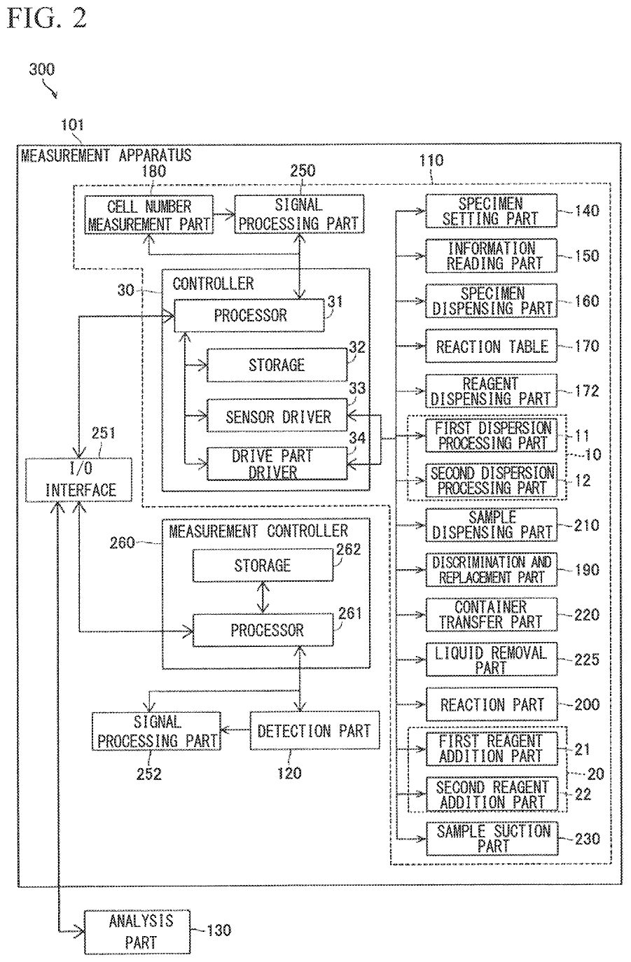

As shown in FIG. 2, the measurement apparatus 101 includes: a controller 30 which controls each component forming the preparation part 110; a signal processing part 250; and an I/O interface 251. Further, the measurement apparatus 101 includes: a measurement controller 260 which controls the detection part 120; and a signal processing part 252.

The controller 30 includes a processor 31, a storage 32, a sensor driver 33, and a drive part driver 34. The storage 32 is implemented by a ROM, a RAM, and the like storing a control program and the like for controlling the preparation part 110. In the storage 32, the dispersion condition 60 (see FIG. 9) for the dispersion process is stored.

The processor 31 of the controller 30 is connected to a processor 261 of the measurement controller 260 via the I/O interface 251. The signal processing part 250 is implemented as a signal processing circuit that performs signal processing on an output signal from the cell number measurement part 180.

The processor 31 of the controller 30 is connected to, via the sensor driver 33 or the drive part driver 34, sensors or drive motors of components (the specimen setting part 140, the information reading part 150, the first dispersion processing part 11, the second dispersion processing part 12, the specimen dispensing part 160, the reaction table 170, the reagent dispensing part 172, the discrimination and replacement part 190, the reaction part 200, the sample dispensing part 210, the container transfer part 220, the liquid removal part 225, the first reagent addition part 21, the second reagent addition part 22, and the sample suction part 230) of the preparation part 110. The processor 31 controls operation of each drive motor on the basis of a detection signal from a corresponding sensor.

The measurement controller 260 includes a processor 261 and a storage 262. The storage 262 is implemented by a ROM, a RAM, and the like storing a control program and data of the detection part 120. The signal processing part 252 is implemented as a signal processing circuit that performs necessary signal processing on an output signal from the detection part 120.

The processor 261 of the measurement controller 260 is connected to the analysis part 130 and the processor 31 of the controller 30 via the I/O interface 251. Accordingly, various types of data can be transmitted and received between the measurement controller 260, the analysis part 130, and the controller 30.

<Details of First Dispersion Processing Part 11>

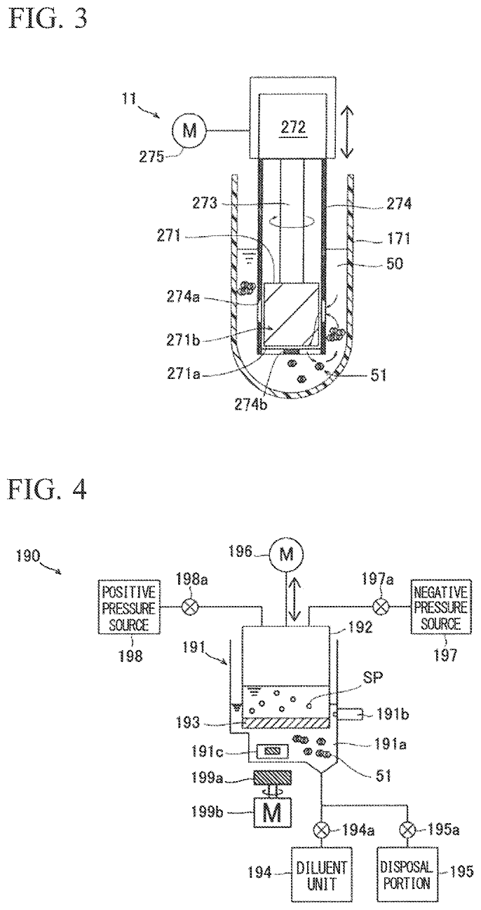

Next, a specific configuration of the first dispersion processing part 11 will be described. As shown in FIG. 3, the first dispersion processing part 11 includes the rotor 271, and a stirring motor 272 which drives the rotor 271 to rotate. An upper end portion of the rotor 271 is connected to an output shaft of the stirring motor 272 via a rotation shaft 273. The first dispersion processing part 11 is configured such that the rotor 271 is inserted and rotated in the specimen 50 in the container 171, whereby aggregated cells contained in the specimen 50 are dispersed into single cells. The first dispersion processing part 11 is controlled by the controller 30.

The first dispersion processing part 11 has a pipe 274 having a cylindrical shape and housing the rotor 271. The pipe 274 has a bottom part, and covers the side face and a lower end face 271a of the rotor 271 with a slight gap from the rotor 271. In the side face of the pipe 274, a hole portion 274a is formed at a position opposed to the side face of the rotor 271. In the bottom face of the pipe 274, a hole portion 274b is formed at a position opposed to the lower end face 271a of the rotor 271. The rotor 271 has a groove 271b in a spiral shape formed in the outer periphery thereof. Through rotation of the rotor 271, the specimen 50 is drawn into the hole portion 274a in the side face of the pipe 274, and the specimen 50 is downwardly sent between the groove 271b of the rotor 271 and the side face of the pipe 274, to be sent between the lower end face 271a of the rotor 271 and the bottom part of the pipe 274.

The lower end face 271a of the rotor 271 is formed in a flat shape. The gap between the lower end face 271a and the inner bottom face of the pipe 274 has a size that does not allow passage of aggregated cells (not less than 100 μm) and that allows passage of a single cell (average 60 μm). Through rotation of the rotor 271, a shearing force is applied to aggregated cells between the inner bottom face of the pipe 274 and the lower end face 271a of the rotor 271. The specimen 50 is sent out from the hole portion 274b in the inner bottom face of the pipe 274. While the rotor 271 is rotating, the specimen 50 is taken in from the hole portion 274a in the side face of the pipe 274, and is circulated so as to be discharged from the hole portion 274b in the bottom part of the pipe 274, and in the course of the circulation, the aggregated cells are dispersed by a shearing force.

The rotor 271, the stirring motor 272, and the pipe 274 are moved in the up-down direction by a raising/lowering motor 275. The first dispersion processing part 11 is downwardly moved by the raising/lowering motor 275 from a position above the container 171 transferred to the first dispersion processing position P4 (see FIG. 1), and inserts the rotor 271 and the pipe 274 into the container 171. After the first dispersion process, the first dispersion processing part 11 is upwardly moved by the raising/lowering motor 275, to withdraw the rotor 271 and the pipe 274 from the inside of the container 171.

<Details of Discrimination and Replacement Part 190>

A configuration of the discrimination and replacement part 190 will be described. As shown in FIG. 4, the discrimination and replacement part 190 includes a filter 193 for separating cells 51. Accordingly, the cells 51 serving as the target of the cell analysis can be effectively separated (filtrated) from unnecessary substances. The discrimination and replacement part 190 is controlled by the controller 30.

Specifically, the discrimination and replacement part 190 includes: the replacement container 191; a piston 192 having a tubular shape and movable in the up-down direction in the replacement container 191; and the filter 193 provided at the lower end face of the piston 192.

The replacement container 191 includes a storage chamber 191a. The storage chamber 191a is provided with a liquid surface detection sensor 191b. Through a channel connected to the bottom face of the storage chamber 191a, the storage chamber 191a is connected to a diluent unit 194 via a valve 194a and is connected to a disposal portion 195 via a valve 195a. The storage chamber 191a is supplied with a diluent (replacement liquid) from the diluent unit 194 via the valve 194a.

The piston 192 causes the liquid to pass through the filter 193 at the bottom part, to separate the specimen 50 into a first liquid mainly containing the measurement target cells (the cells 51) and a second liquid mainly containing cells SP having a diameter smaller than that of the measurement target cells. That is, the second liquid having passed through the filter 193 is stored in the piston 192, and the first liquid remains on the storage chamber 191a side of the replacement container 191.

The discrimination and replacement part 190 includes a motor 196 which moves the piston 192 up and down. A predetermined amount of the specimen 50 is dispensed into the replacement container 191 by the sample dispensing part 210. The piston 192 is moved downwardly toward the bottom part of the replacement container 191 by the motor 196. In the piston 192, a negative pressure source 197 is connected via a valve 197a, and a positive pressure source 198 is connected via a valve 198a.

When the valve 197a is opened, a negative pressure is supplied to the inside of the piston 192. Then, a liquid (the first liquid) containing the measurement target cells (the cells 51) remains below the filter 193 in the replacement container 191, and a liquid (the second liquid) containing the cells SP having a diameter smaller than that of the measurement target cells is stored in the inside of the piston 192.

The liquid surface detection sensor 191b detects the liquid surface of the first liquid in the replacement container 191. The liquid surface detection sensor 191b is a capacitance-type sensor. The liquid surface detection sensor 191b is positioned slightly above the lower face of the filter 193 at the lowest point of the piston 192. When the liquid surface detection sensor 191b has detected the liquid surface, the valve 197a is closed, and liquid suction to the inside of the piston 192 due to the negative pressure is stopped. Then, the valve 198a is opened, a positive pressure is supplied to the inside of the piston 192, and the measurement target cells attached to the lower face of the filter 193 are returned to the storage chamber 191a.

The other small cells SP are discriminated (separated) from the measurement target cells (the cells 51) by the filter 193. As a result of the liquid suction through the piston 192, the liquid component in the specimen 50 is replaced from the preservation liquid to the diluent. The discrimination and replacement process is performed a plurality of times. That is, a series of operations of: upward movement of the piston 192; supply of the diluent; downward movement of the piston 192 and liquid suction (supply of a negative pressure); liquid surface detection (stop of supply of the negative pressure); and supply of a positive pressure is performed a plurality of times.

The storage chamber 191a has stored therein a rotor 191c having a magnet built therein. Below the bottom part of the storage chamber 191a, a magnet 199a for applying a magnetic force to the rotor 191c and a motor 199b for rotating the magnet 199a are provided. Through rotation of the rotor 191c, the measurement target cells attached to the lower face of the filter 193 are detached, and the measurement target cells (the cells 51) are collected to an outer peripheral portion of the storage chamber 191a due to the centrifugal force of the rotating first liquid.

The first liquid containing the measurement target cells (the cells 51) collected to the outer peripheral portion of the storage chamber 191a is suctioned by the sample dispensing part 210. Accordingly, a measurement sample having a high concentration of the measurement target cells can be obtained.

<Details of Second Dispersion Processing Part 12>

Next, a specific configuration of the second dispersion processing part 12 will be described. As shown in FIG. 5, the second dispersion processing part 12 includes: a liquid storage portion 281 storing a liquid 281a such as water; and an ultrasonic vibrator 282 for applying, through the liquid 281a, ultrasonic vibration WV to the specimen 50 in the second container 202.

The liquid storage portion 281 has a recessed shape so as to store the liquid 281a, and is formed in a substantially cylindrical shape. At an opening in an upper end portion of the liquid storage portion 281, a lid 283 having formed therein a circular hole having a size that allows insertion of a second container 202 therethrough is provided. With this lid 283, scattering of the liquid 281a associated with the ultrasonic vibration WV to the outside is suppressed.

The ultrasonic vibrator 282 is disposed below the liquid storage portion 281. The ultrasonic vibrator 282 is implemented as a Langevin-type vibrator, for example. The Langevin-type vibrator has a structure in which a piezoelectric element 282b is interposed between a pair of vibration plates 282a, and generates vibration in the thickness direction (the direction in which the pair of vibration plates 282a face each other) due to application of alternating voltage to the piezoelectric element 282b. The peripheral wall of the liquid storage portion 28 having a substantially cylindrical shape is provided with, from the bottom in order, a liquid communication hole 281b, an upper outlet hole 281c, and an overflow channel (hole) 281d. The liquid communication hole 281b is in communication with, via a channel switching valve 287, a liquid supply source 284 and a liquid chamber 286 connected to a suction source 285.

When the liquid 281a is supplied, the liquid supply source 284 is caused to be communicated with the liquid communication hole 281b by the channel switching valve 287, whereby the liquid 281a is supplied from the liquid supply source 284 to the liquid storage portion 281. The upper outlet hole 281c is in communication with the liquid chamber 286, and has a function of discharging the liquid 281a in the liquid storage portion 281. The height (the upper limit position) of the liquid surface of the liquid 281a in the liquid storage portion 281 is determined by the position where the upper outlet hole 281c is formed.

The overflow channel 281d is provided to the upper side of the upper outlet hole 281c, and is in communication with the liquid chamber 286. When the liquid 281a is excessively supplied to exceed the upper outlet hole 281c, or when the second container 202 is excessively inserted into the liquid storage portion 281, the liquid 281a is discharged from the overflow channel 281d to the liquid chamber 286.

The suction source 285 serving as a negative pressure source is connected to the liquid chamber 286, and the liquid in the liquid storage portion 281 can be drawn into the liquid chamber 286.

In the second dispersion process by the second dispersion processing part 12, the second container 202 is immersed into the liquid 281a in the liquid storage portion 281, in a state where the second container 202 is gripped by the grip part 221 of the container transfer part 220. In this state, the ultrasonic vibrator 282 is driven and the ultrasonic vibration WV is generated.

In the second dispersion process, the ultrasonic vibration WV generated by the ultrasonic vibrator 282 is propagated, through the liquid 281a in the liquid storage portion 281 and the second container 202, to the specimen 50 in the second container 202. The ultrasonic vibration WV causes cavitation (generation of fine bubbles and bursting of bubbles) in the specimen 50 and aggregated cells are caused to be dispersed due to an impact (pressure change) associated with the cavitation. Accordingly, the aggregated cells (the measurement target cells) remaining after the first dispersion process in the specimen 50 in the second container 202 are dispersed into single cells.

<Details of Detection Part 120 and Cell Number Measurement Part 180>

FIG. 6 is a block diagram showing a configuration example of the detection part 120 of the measurement apparatus 101. In the example in FIG. 6, the detection part 120 is implemented as a flow cytometer. The detection part 120 includes a light source 121 implemented as a semiconductor laser light source, and laser light emitted from the light source 121 passes through a lens system 122 and is condensed on a measurement sample flowing in the flow cell 123. Forward scattered light generated by this laser light from each cell in the measurement sample passes through an objective lens 124a and an optical filter 124b, and is detected by a photodiode 125 serving as a light receiver. The lens system 122 is composed of lenses including a collimator lens, a cylinder lens, a condenser lens, and the like.

Further, fluorescence and side scattered light generated from the cell passes through an objective lens 126a disposed sideways relative to the flow cell 123 and enters a dichroic mirror 126b. The fluorescence and the side scattered light reflected by the dichroic mirror 126b enter a dichroic mirror 126c.

The fluorescence having passed through the dichroic mirror 126c passes through an optical filter 126d, and is detected by a photomultiplier tube 127. The side scattered light reflected by the dichroic mirror 126c passes through an optical filter 126e, and is detected by a photomultiplier tube 128.

The photodiode 125, the photomultiplier tube 127, and the photomultiplier tube 128 convert the detected lights into electric signals, and output a forward scattered light signal (FSC), a side scattered light signal (SSC), and a fluorescence signal (SFC), respectively. These signals are sent to the signal processing part 252 (see FIG. 2).

As shown in FIG. 2, optical information (forward scattered light waveform data (FS), side scattered light waveform data (SS), and fluorescence waveform data (FL)) obtained through signal processing such as a filtering process, an A/D conversion process, and the like in the signal processing part 252 is used in calculation of data for cell analysis in the measurement controller 260 (the processor 261). The calculated data is sent to the analysis part 130 via the I/O interface 251.

In the example in FIG. 6, the measurement apparatus 101 is provided with an imaging part 290 in addition to the detection part 120. The imaging part 290 includes a light source 291 implemented as a pulsed laser, and a CCD camera 292. Laser light from the light source 291 passes through a lens system 293, enters the flow cell 123, further passes through the objective lens 126a and the dichroic mirror 126b, and forms an image at the CCD camera 292. The light source 121 emits light at a predetermined timing, thereby enabling imaging by the CCD camera 292.

An image of each cell captured by the CCD camera 292 is sent by the measurement controller 260 (the processor 261) shown in FIG. 2 to the analysis part 130 via the I/O interface 251. The image of each cell is stored into the analysis part 130 so as to correspond to data for cell analysis for the cell.

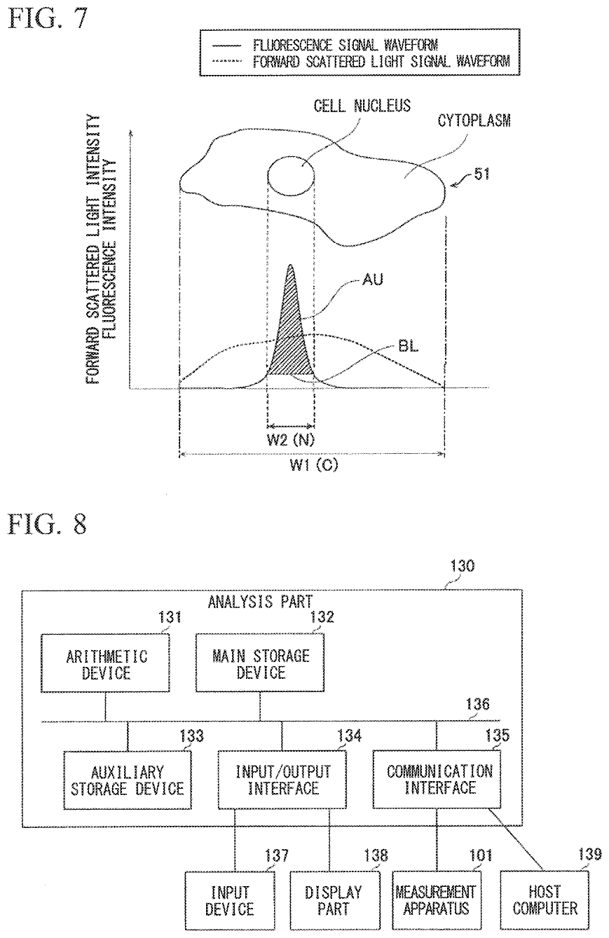

FIG. 7 shows a schematic diagram of a cell including a cytoplasm and a cell nucleus, and a forward scattered light signal waveform and a fluorescence signal waveform obtained from this cell. In FIG. 7, the vertical axis of the graph represents intensity of the forward scattered light and the fluorescence. A width W1 of the forward scattered light signal waveform indicates the width of the cytoplasm, i.e., the value indicating the size of the cell (C). A width W2 of the fluorescence signal waveform indicates the value of the size of the cell nucleus (N).

The measurement controller 260 calculates data indicating the size of the cell 51 (C), the size of the cell nucleus (N) of the cell 51, and the DNA content of the cell 51, from the optical information. The measurement controller 260 calculates the size of the cell 51 (C) and the size of the cell nucleus (N) on the basis of the width W1 (the pulse width of the forward scattered light signal) of the forward scattered light signal waveform generated from each cell, and the width W2 (the pulse width of the fluorescence signal) generated from the cell nucleus of the cell. Further, the measurement controller 260 calculates the DNA content of the cell 51 on the basis of the area (the pulse area of the fluorescence signal) of a region AU (the hatched portion in FIG. 7) surrounded by the fluorescence signal waveform (fluorescence signal) and a predetermined baseline BL with respect to the fluorescence signal waveform in the graph shown in FIG. 7. Instead of the area of the region AU surrounded by the fluorescence signal waveform and the predetermined baseline BL, the height of the fluorescence signal waveform may be used to calculate the DNA content.

With reference back to FIG. 6, the cell number measurement part 180 adopts a flow cytometer having a configuration substantially the same as that of the detection part 120 above. Therefore, detailed description of the cell number measurement part 180 is omitted. Since the cell number measurement part 180 preliminarily performs measurement of the number (concentration) of the measurement target cells before the main measurement performed by the detection part 120, it is sufficient that the cell number measurement part 180 can output a signal for counting the number of cells (it is sufficient that the forward scattered light signal (FSC) can be obtained). The cell number measurement part 180 need not be provided with an imaging part.

<Details of Analysis Part 130>

FIG. 8 is a block diagram showing an example of a configuration of a computer usable as the analysis part 130. The computer includes an arithmetic device 131, a main storage device 132, an auxiliary storage device 133, an input/output interface 134, and a communication interface, which are connected to each other via a bus 136. The arithmetic device 131, the main storage device 132, and the auxiliary storage device 133 may each be a processor, a RAM, or a hard disk drive. The input/output interface 134 has connected thereto an input device 137 with which the user inputs various types of information to the computer, and a display part 138 on which the computer displays various types of information. The input device 137 and the display part 138 may be built in the computer, or may be (externally) connected to the computer. For example, the input device 137 may be a keyboard, a mouse, a touch sensor, or the like. The display part 138 is a display device such as a liquid crystal monitor. The display part 138 may be a touch panel display integrated with a touch sensor as the input device 137. The communication interface 135 is an interface through which the analysis part 130 performs communication with an external device including the measurement apparatus 101 and a host computer 139.

The auxiliary storage device 133 has stored therein various types of programs for causing the computer to operate as the analysis part 130. The arithmetic device 131 extracts programs stored in the auxiliary storage device 133 onto the main storage device 132, and executes commands included in the programs, thereby causing the computer to function as the analysis part 130.

The analysis part 130 analyzes a measurement item on the basis of the size of the cell, the size of the cell nucleus, and the DNA content which are obtained from optical information detected by the detection part 120. In the analysis based on morphological information about each individual cell 51 such as the size of the cell 51, the size of the cell nucleus, and the DNA content, it is particularly important to form each cell 51 in a state of a single cell, without destroying the cell 51. Therefore, it is particularly useful to suppress variation in the detection result through a dispersion process under an appropriate dispersion condition.

Using the size of the cell 51, the size of the cell nucleus, the DNA content, and a parameter calculated through a combination of these, the arithmetic device 131 creates a scattergram having a first axis representing a parameter indicating the size of each cell and a second axis representing a ratio (N/C ratio) between the size of the cell nucleus (N) and the size of the cell (C), and counts the number of cells plotted in a predetermined region in the scattergram. Then, in the created scattergram, a borderline dividing the scattergram into two regions having different values of the N/C ratio is set. Then, an analysis result is created by using a ratio (CPIx value) between the number of cells having a DNA content not greater than a predetermined threshold, and the number of cells having a DNA content not less than the predetermined threshold, in the region on the side where the N/C ratio has higher values with respect to this borderline. Here, the CPIx value is a value indicating a ratio of cells having a DNA content greater than the DNA content of a diploid cell (a cell having a DNA content equivalent to that of a normal cell in the G0 phase or the G1 phase). A high CPIx value is utilized as a value that indicates how much the presence of a cell having cancerated and a cell having become heteromorphic is suspected. The analysis result is judged, for example, on the basis of whether or not the number of cells in a region defined for each measurement item in the scattergram exceeds a threshold for the measurement item, and has information regarding positive (exceeding the threshold) or negative (not greater than the threshold). The analysis according to the CPIx value using the above parameter is disclosed in US Patent Application Publication No. 2019/0086317. The analysis according to the CPIx value using the above parameter is incorporated herein by reference as if shown herein.

<Details of Dispersion Condition>

Next, a dispersion condition corresponding to the type of the specimen 50 will be described. In the present embodiment, the controller 30 is configured to obtain a dispersion condition corresponding to the type of the specimen 50 and control the dispersion processing part 10 (the first dispersion processing part 11 and the second dispersion processing part 12) so as to perform the dispersion processes on the specimen according to the obtained dispersion condition.

That is, the controller 30 performs control of causing the preparation part 110 to execute the sample preparation method according to the present embodiment. The sample preparation method includes: a step of obtaining a dispersion condition corresponding to the type of the specimen 50; and a step of dispersing aggregated cells 51 contained in the specimen 50, according to the obtained dispersion condition.

Therefore, even when the type of the specimen 50 is changed and thus the aggregation state of the cells 51 that are contained in the specimen 50 and that should be dispersed is changed, the dispersion process can be performed under an appropriate dispersion condition corresponding to the type of the specimen 50. That is, since a dispersion process suitable for the aggregation state of the cells 51 can be performed according to a dispersion condition corresponding to the type of the specimen 50, each aggregated cell population can be dispersed into a larger number of single cells. As a result, cell analysis can be performed by using the sample 59 prepared so as to have an appropriate cell dispersion state, and thus, automation of highly accurate analysis of cells contained in different types of specimens collected from different organs or sites can be realized.

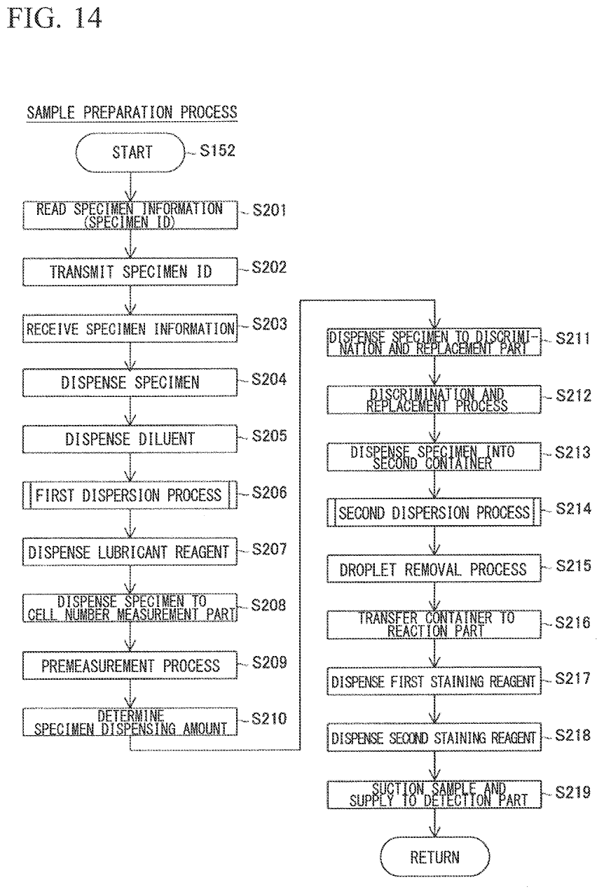

In the present embodiment, the step of dispersing the cells 51 includes at least one of a step of dispersing aggregated cells 51 by applying a shearing force to the specimen 51, and a step of dispersing aggregated cells 51 by applying ultrasonic vibration to the specimen 50. In the configuration example shown in FIG. 1, both of the step (the first dispersion process, see step S206 in FIG. 14) of dispersing aggregated cells 51 by applying a shearing force to the specimen 50 by the first dispersion processing part 11 and the step (the second dispersion process, see step S214 in FIG. 14) of dispersing aggregated cells 51 by applying ultrasonic vibration to the specimen 50 by the second dispersion processing part 12 are performed.

Therefore, in the step of applying a shearing force to the specimen 50, the shearing force is caused to mechanically act on each aggregated cell population, whereby the aggregated cell population can be dispersed. In the step of applying ultrasonic vibration to the specimen 50, the aggregated cell population can be dispersed (separated) by the vibration. Only one of the step of applying a shearing force to the specimen 50 and the step of applying ultrasonic vibration to the specimen 50 may be performed.

In the example shown in FIG. 9, the dispersion condition 60 includes a first dispersion condition 61 and a second dispersion condition 62 each corresponding to the type of the specimen. Each of the first dispersion condition 61 and the second dispersion condition 62 includes: a dispersion condition 63 for the step (the first dispersion process) of applying a shearing force to the specimen 50; and a dispersion condition 64 for the step (the second dispersion process) of applying ultrasonic vibration to the specimen 50.

The dispersion condition 63 for the step (the first dispersion process) of applying a shearing force to the specimen 50 includes a processing time 63a and a speed 63b of a member that applies the shearing force. The speed 63b of the member that applies the shearing force is the number of revolutions of the stirring motor 272 which drives the rotor 271 of the first dispersion processing part 11. Therefore, the processing time 63a of dispersion and the speed 63b (which influences the magnitude of the shearing force) of the rotor 271 applying the shearing force can be appropriately set in accordance with the specimen 50. As a result, even when the type of the specimen 50 is changed, each aggregated cell population can be dispersed while the cells 51 are suppressed from being destroyed. In the example in FIG. 9, the processing time 63a is a parameter defined by two variables, i.e., a rotation time and the number of times of processing.

The dispersion condition 64 for the step of applying ultrasonic vibration to the specimen 50 includes a processing time 64a, a frequency of vibration 64b, and an intensity 64c of ultrasonic vibration. In the example in FIG. 9, the intensity 64c of ultrasonic vibration is the output (drive power value) of the ultrasonic vibrator 282 of the second dispersion processing part 12. Therefore, the processing time 64a of dispersion, the frequency of vibration 64b, and the intensity 64c of ultrasonic vibration can be appropriately set in accordance with the specimen 50. As a result, even when the type of the specimen 50 is changed, the aggregated cells can be dispersed while the cells 51 are suppressed from being destroyed.

As shown in FIG. 9, the dispersion condition 60 is set in accordance with the organ and/or site from which the specimen 50 is derived, i.e., the type of the specimen 50. In the step of obtaining a dispersion condition, the controller 30 obtains the first dispersion condition 61 or the second dispersion condition 62 that corresponds to the organ and/or site from which the specimen 50 is derived. Accordingly, the first dispersion condition 61 or the second dispersion condition 62 suitable for dispersing the cells 51 that are present in a large number in the organ and/or site can be obtained. Since the shape and the manner of aggregation of the cells 51 are different depending on the organ or site, if the specimen 50 is dispersed under an appropriate dispersion condition 60, a sample suitable for cell analysis can be prepared from the specimen 50 derived from various organs and/or sites.

The specimen 50 is a biological sample selected from a site of one of the uterine cervix, the uterine body, the oral cavity, the esophagus, and the bronchus. Thus, sample preparation suitable for analysis of the cells 51 contained in various specimens 50 can be performed. In the example in FIG. 9, two types of dispersion conditions, i.e., the first dispersion condition 61 (condition No. 1), and the second dispersion condition 62 (condition No. 2), are each set in advance in accordance with the organ and/or site from which the specimen 50 is derived. The dispersion condition 60 set in advance is stored in the storage 32 (see FIG. 2) of the controller 30. As described later, the controller 30 obtains, from the storage 32, the dispersion condition 60 corresponding to the specimen, on the basis of the specimen information 90.

Here, in the example in FIG. 9, in a case of a specimen 50 containing more stratified squamous epithelial cells 55 (see FIG. 10) than simple columnar epithelial cells 56 (see FIG. 10), the first dispersion condition 61 (condition No. 1) is obtained. In a case of a specimen 50 containing more simple columnar epithelial cells 56 (see FIG. 10) than stratified squamous epithelial cells 55 (see FIG. 10), the second dispersion condition 62 (condition No. 2) is obtained. The second dispersion condition 62 is a condition having a dispersion effect higher than that of the first dispersion condition 61. As shown in FIG. 10, an epithelial tissue of the uterine cervix corresponding to the first dispersion condition 61 is mainly composed of stratified squamous epithelial cells 55. As shown in FIG. 10, an epithelial tissue of the uterine body corresponding to the second dispersion condition 62 is mainly composed of simple columnar epithelial cells 56. As shown in FIG. 9, the second dispersion condition 62 is set to have a processing time 64a of the second dispersion process longer than that of the first dispersion condition 61, and thus is a condition having a high dispersion effect, accordingly.

Thus, in the step of obtaining a dispersion condition, when the specimen 50 is a tissue of the uterine cervix, the first dispersion condition 61 is obtained, and when the specimen 50 is a tissue of the uterine body, the second dispersion condition 62 having a processing time 64a for applying ultrasonic vibration to the specimen 50 longer than that of the first dispersion condition 61 is obtained by the controller 30. When the specimen 50 is a tissue of the uterine cervix, the controller 30 obtains the first dispersion condition 61, and controls the dispersion processing part 10 (the first dispersion processing part 11 and the second dispersion processing part 12) so as to perform the dispersion processes on the specimen 50 according to the obtained first dispersion condition 61. When the specimen 50 is a tissue of the uterine body, the controller 30 obtains the second dispersion condition 62 and controls the dispersion processing part 10 (the first dispersion processing part 11 and the second dispersion processing part 12) so as to perform the dispersion processes on the specimen 50 according to the obtained second dispersion condition 62.

Through the result of studies by the present inventors, it was found that, as for simple columnar epithelial cells 56 which are present in a large number in the uterine body, each aggregated cell population is less likely to be dispersed into single cells when compared with stratified squamous epithelial cells 55 which are present in a large number in the uterine cervix and the like. Therefore, by causing the second dispersion condition 62 to have a higher dispersion effect than that of the first dispersion condition 61 (i.e., by increasing the processing time 64a for applying ultrasonic vibration), it is possible to perform an effective dispersion process also on the specimen 50 containing a large number of simple columnar epithelial cells 56. Accordingly, variation in the detection result with respect to the specimen 50 derived from the uterine body can be suppressed, and cell analysis can be realized. A dispersion condition for a specimen 50 containing epithelial cells (simple columnar epithelial cells 56) of the uterine body as the measurement target will be described in detail in Examples described later.

The controller 30 causes at least one of the first dispersion processing part 11 and the second dispersion processing part 12 to operate under the dispersion condition 60 corresponding to the type of the specimen 50. In the example in FIG. 9, the first dispersion process by the first dispersion processing part 11 is common between condition No. 1 and condition No. 2. That is, the controller 30 controls the first dispersion processing part 11 so as to operate under the same dispersion condition between a case where the specimen 50 is a tissue of the uterine cervix and a case where the specimen 50 is a tissue of the uterine body. Meanwhile, the second dispersion process by the second dispersion processing part 12 is different between condition No. 1 and condition No. 2 in accordance with the type of the specimen 50. The controller 30 controls the second dispersion processing part 12 so as to operate under different dispersion conditions between a case where the specimen 50 is a tissue of the uterine cervix and a case where the specimen 50 is a tissue of the uterine body. The significance of setting only the dispersion condition for the second dispersion process in accordance with the specimen 50 will be described in Examples described later.

<Step of Obtaining Dispersion Condition 60>

The step of obtaining a dispersion condition will be described. The step of obtaining the dispersion condition 60 corresponding to the type of the specimen 50 is performed by the controller 30 (see FIG. 2) of the preparation part 110.

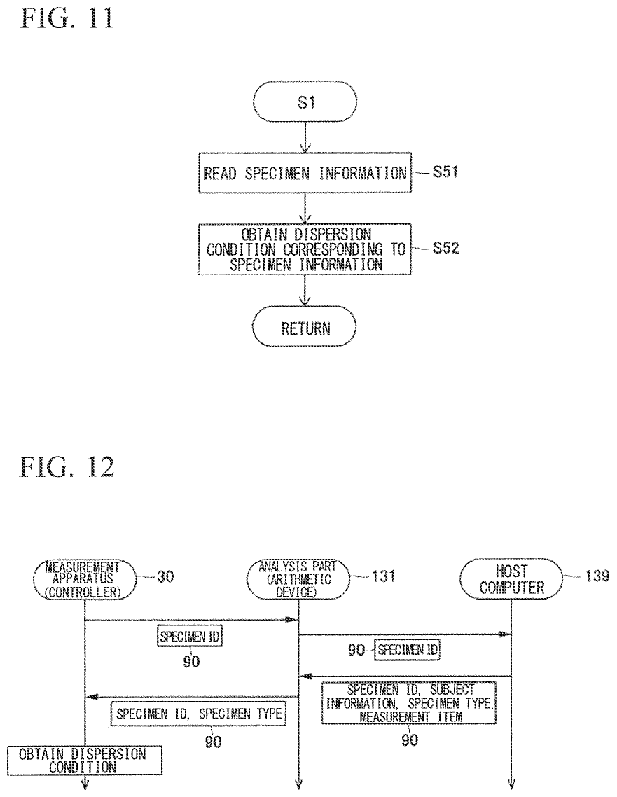

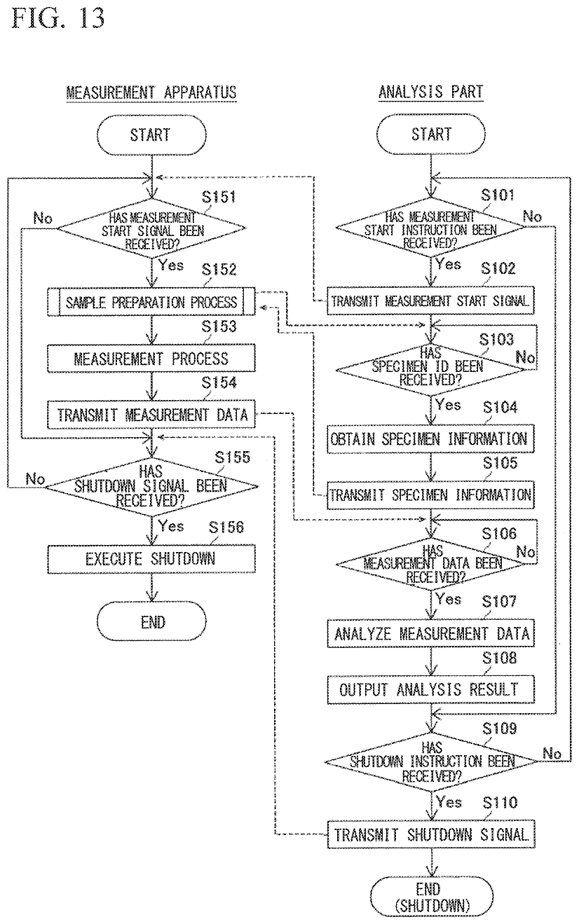

In the example in FIG. 11, the step of obtaining the dispersion condition 60 includes: a step S51 of obtaining specimen information 90 (see FIG. 12) regarding the specimen 50; and a step S52 of obtaining the dispersion condition 60 corresponding to the organ and/or site from which the specimen 50 is derived, on the basis of the obtained specimen information 90. The controller 30 of the preparation part 110 obtains the dispersion condition 60 corresponding to the organ and/or site from which the specimen 50 is derived, on the basis of the specimen information 90 read by the information reading part 150. Since the specimen information 90 is obtained, the organ and/or site from which the specimen 50 is derived can be easily identified. As a result, an appropriate dispersion condition 60 corresponding to the type of the specimen 50 can be assuredly obtained.

The controller 30 obtains a specimen ID included in the specimen information 90 from the information reading part 150 (see FIG. 1). As shown in FIG. 12, the controller 30 transmits the obtained specimen ID to the analysis part 130 via the I/O interface 251 (see FIG. 2). The arithmetic device 131 of the analysis part 130 transmits the specimen ID to the host computer 139 on a network via the communication interface 135 (see FIG. 8), and obtains remaining specimen information 90 corresponding to the specimen ID from the host computer 139. The specimen information 90 that is obtained includes subject information regarding the subject from whom the specimen has been collected, the type of the specimen 50, and a measurement item with respect to the specimen 50. The type of the specimen 50 is information of the organ and/or site from which the specimen 50 is derived. The controller 30 obtains the type of the specimen 50 from the analysis part 130.

The controller 30 obtains the first dispersion condition 61 or the second dispersion condition 62 that corresponds to the type of the specimen 50, from the storage 32 (see FIG. 2) having stored therein a database shown in FIG. 9. As shown in FIG. 9, for example, when the specimen 50 is “epithelial cells of the uterine cervix”, the controller 30 obtains, from the storage 32, the dispersion conditions 63 and 64 set in the first dispersion condition 61 of “No. 1” corresponding to the specimen information 90. For example, when the specimen 50 is “epithelial cells of the uterine body”, the controller 30 obtains the dispersion conditions 63 and 64 set in the second dispersion condition 62 of “No. 2” corresponding to the specimen information 90.

Meanwhile, the effect of the dispersion process can be influenced by the number of cell populations (the concentration of cells) contained in the specimen 50 per unit amount. Therefore, the controller 30 obtains, from the cell number measurement part 180, information about the number of cell populations contained in the specimen 50 having been subjected to the first dispersion process performed by the first dispersion processing part 11.

On the basis of the information (concentration information) of the number of cell populations contained in the specimen 50, the controller 30 determines a dispensing amount of the specimen 50 such that the number of cell populations contained in the specimen 50 to be dispensed to the discrimination and replacement part 190 is in a predetermined allowable range. When the number of cell populations per unit amount is larger (the concentration is higher), the controller 30 sets the dispensing amount of the specimen 50 to be relatively smaller. When the number of cell populations per unit amount is smaller (the concentration is lower), the controller 30 sets the dispensing amount of the specimen 50 to be relatively larger. In this manner, with respect to any specimen 50, the controller 30 determines a dispensing amount of the specimen 50 such that the number of cell populations of the measurement target cells to be supplied to the discrimination and replacement part 190 is generally constant (within the allowable range). The second dispersion process by the second dispersion processing part 12 is performed on the specimen 50 having been processed by the discrimination and replacement part 190.

As a result, even if the cell concentration in the original specimen 50 (stored in the specimen container 141) is different, the number of cell populations in the specimen 50 to be subjected to the second dispersion process is adjusted so as to be within the allowable range. Therefore, variation in the dispersion effect due to difference in the cell concentration of the specimen 50 is reduced.

(Analysis Operation of Cell Analyzer 300)

Next, with reference to FIG. 13 to FIG. 16, an analysis operation of the cell analyzer 300 will be described. Control of operations of the detection part 120 and the signal processing part 252 of the measurement apparatus 101 is performed by the measurement controller 260 (the processor 261), and control of operations of the cell number measurement part 180, the signal processing part 250, and the preparation part 110 of the measurement apparatus 101 is performed by the controller 30 (the processor 31). Control of the analysis part 130 is performed by the arithmetic device 131. Hereinafter, the configurations of components of the cell analyzer 300 will be referred to as in FIG. 1 and FIG. 2.

In analysis by the cell analyzer 300, first, a specimen container 141 storing a specimen 50 that contains cells 51 and a preservation liquid having methanol as a main component is set to the specimen setting part 140 (see FIG. 1) by the user.

In step S101, the arithmetic device 131 of the analysis part 130 determines whether or not a measurement start instruction has been received from the user. The arithmetic device 131 receives an input through a measurement start button on a menu screen displayed on the display part 138, for example. When having received the measurement start instruction, the arithmetic device 131 of the analysis part 130 transmits a measurement start signal to the measurement apparatus 101 in step S102.

In step 151, the controller 30 of the measurement apparatus 101 determines whether or not the measurement start signal has been received. When having received the measurement start signal, the controller 30 starts a sample preparation process in in step S152.

<Sample Preparation Process>

The controller 30 of the measurement apparatus 101 controls the specimen setting part 140 so as to transport the specimen rack 142 holding the specimen 50 to the specimen suction position P1. At this time, as shown in FIG. 14, in step S201, the controller 30 controls the information reading part 150 to execute reading of the specimen information 90 (the specimen ID) from the information storage medium 151 attached to the specimen container 141.

In step S202, the controller 30 transmits the read specimen ID to the analysis part 130. As shown in FIG. 13, the arithmetic device 131 of the analysis part 130 determines, in step S103, whether or not the specimen ID has been received. The arithmetic device 131 repeats the determination until receiving the specimen ID. When having received the specimen ID, the arithmetic device 131 obtains, in step S104, remaining specimen information 90 associated with the specimen ID, by using the specimen ID. The arithmetic device 131 transmits the specimen ID to the host computer 139 on the network via the communication interface 135, and obtains, from the host computer 139, the specimen information 90 (subject information, the type of the specimen 50, a measurement item) corresponding to the specimen ID.

In step S105, the arithmetic device 131 transmits the specimen information 90 (the type of the specimen 50) to the measurement apparatus 101 (the controller 30) via the communication interface 135. Accordingly, in step S203 in FIG. 14, the controller 30 of the measurement apparatus 101 receives the specimen information 90 (the type of the specimen 50) from the analysis part 130.

In step S204, the controller 30 of the measurement apparatus 101 controls the specimen dispensing part 160 so as to suction the specimen 50 from the specimen container 141 disposed at the specimen suction position P1 of the specimen setting part 140 and dispense the specimen 50 into a container 171 in the reaction table 170. The specimen dispensing part 160 dispenses a predetermined amount of the specimen 50 into the container 171 at the specimen discharge position P2. The controller 30 controls the reaction table 170 so as to intermittently rotate by a predetermined angle every predetermined time.

In step S205, the controller 30 controls the reagent dispensing part 172 so as to dispense a predetermined amount of the diluent into the container 171 to which the specimen 50 has been dispensed and which has been transferred to the reagent dispensing position P3.

In step S206, the controller 30 controls the first dispersion processing part 11 so as to perform the first dispersion process on the specimen 50 in the container 171 to which the diluent has been dispensed.

<First Dispersion Process>

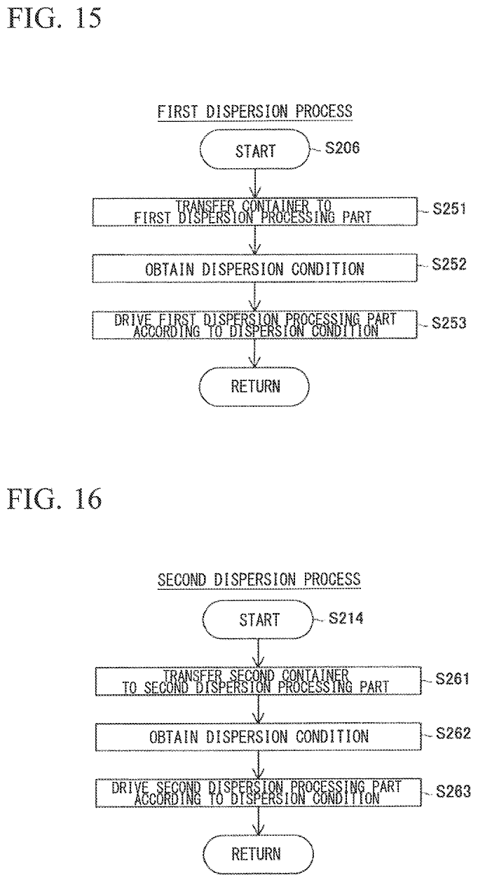

As shown in FIG. 15, the controller 30 controls, in step S251, the reaction table 170 so as to transfer the container 171 to the first dispersion processing position P4 through rotation of the reaction table 170.

In step S252, the controller 30 reads out, from the storage 32, the dispersion condition 63 (see FIG. 9) for the first dispersion process set in the first dispersion condition 61 or the second dispersion condition 62 in accordance with the type of the specimen 50.

In step S253, the controller 30 performs control of driving the first dispersion processing part 11 (see FIG. 3) in accordance with the obtained dispersion condition 63. The controller 30 causes the rotor 271 of the first dispersion processing part 11 to be downwardly moved into the container 171. Then, the controller 30 performs control of driving the rotor 271 (the stirring motor 272) so as to rotate at the speed 63b (the number of revolutions) and for the processing time 63a (time and the number of times) according to the dispersion condition 63 for the first dispersion process.

Upon completion of the rotation drive performed the set number of times, the first dispersion process is completed. The controller 30 causes the rotor 271 of the first dispersion processing part 11 to be upwardly moved to be outside the container 171.

With reference back to FIG. 14, the controller 30 controls, in step S207, the reagent dispensing part 172 so as to dispense the lubricant reagent into the container 171 having been subjected to the first dispersion process.

In step S208, the controller 30 controls the reagent dispensing part 172 so as to dispense, to the cell number measurement part 180, the specimen 50 into which the lubricant reagent has been dispensed after the first dispersion process. The controller 30 controls the reagent dispensing part 172 so as to suction the specimen 50 in the container 171 transferred to the reagent dispensing position P3 and dispense a predetermined amount of the suctioned specimen 50 at the dispensing position P6 for the cell number measurement part 180.

In step S209, the controller 30 performs control regarding measurement of the number of cell populations performed by the cell number measurement part 180. The controller 30 causes the dispersed specimen 50 to be sent to the flow cell (see FIG. 6) of the cell number measurement part 180. Then, the cell number measurement part 180 performs premeasurement (detection of the number of cell populations of the measurement target cells contained in the specimen 50) of the specimen 50. The controller 30 obtains the number of cell populations per unit amount of the specimen 50 via the signal processing part 250. That is, concentration information reflecting the concentration of the measurement target cells contained in the specimen 50 is obtained.

In step S210, the controller 30 determines a dispensing amount of the specimen 50 to be dispensed to the discrimination and replacement part 190. The controller 30 calculates the dispensing amount of the specimen 50 on the basis of the concentration (the number of measurement target cells per unit volume) of the measurement target cells in the specimen 50 used in the premeasurement and the allowable range of the number of cells. The controller 30 calculates the dispensing amount of the specimen 50 such that: a significant number of cells is ensured; and the number of cell populations is in the allowable range suitable for the second dispersion process performed by the second dispersion processing part 12.

In step S211, the controller 30 controls the sample dispensing part 210 so as to supply, to the discrimination and replacement part 190, the dispensing amount of the specimen 50 determined in step S210. The controller 30 controls the sample dispensing part 210 so as to suction the specimen 50 in the container 171 transferred to the sample suction position P5 and dispense the suctioned specimen 50 to the discrimination and replacement part 190.

In step S212, the controller 30 controls the discrimination and replacement part 190 so as to execute the discrimination and replacement process.

In this discrimination and replacement process, the preservation liquid having alcohol as a main component in the specimen 50 is replaced with the diluent and the measurement target cells (the cells 51) are discriminated from the other cells SP, in the discrimination and replacement part 190 (see FIG. 4). In the course of the discrimination, the first liquid containing the measurement target cells is condensed, and the concentration of the measurement target cells is increased. As a result, a condensed liquid that contains a necessary significant number of cells and in which the measurement target cells are condensed and so as to contain a number of cell populations suitable for the second dispersion process, is obtained.

In step S213, the controller 30 performs control of causing the specimen 50 having been subjected to the discrimination and replacement process, to be dispensed from discrimination and replacement part 190 into a second container 202. The controller 30 controls the container transfer part 220 so as to grip and take out the second container 202 set in a holder 201 in the reaction part 200 and position the second container 202 at the dispensing position P8 for the second container 202. Further, the controller 30 controls the sample dispensing part 210 so as to suction the specimen 50 having been subjected to the discrimination and replacement process from the discrimination and replacement part 190 and dispense the suctioned specimen 50 into the second container 202 held in the container transfer part 220. The controller 30 controls the sample dispensing part 210 so as to dispense a predetermined amount of the specimen 50. Since the number of the cells 51 in the specimen 50 has been adjusted, when the predetermined amount of the specimen 50 determined in advance is dispensed, a desired number of the measurement target cells is stored in the second container 202.

In step S214, the controller 30 controls the second dispersion processing part 12 so as to perform the second dispersion process.

<Second Dispersion Process>

As shown in FIG. 16, in step S261, the controller 30 controls the container transfer part 220 so as to transfer, to the second dispersion processing part 12, the second container 202 storing the specimen 50 having been subjected to the discrimination and replacement process. The container transfer part 220 is controlled so as to downwardly move the second container 202 through the circular hole in the lid 283 of the second dispersion processing part 12 (see FIG. 5) to the inside of the liquid storage portion 281, thereby inserting the bottom part of the second container 202.

In step S262, the controller 30 reads out and obtains, from the storage 32, the dispersion condition 64 for the second dispersion process corresponding to the type of the specimen.

In step S263, the controller 30 performs control of driving the second dispersion processing part 12 in accordance with the obtained dispersion condition 64 (here, No. 1 or No. 2). That is, the controller 30 drives the ultrasonic vibrator 282 in accordance with the intensity 64c (output), the frequency of vibration 64b, and the processing time 64a specified by the dispersion condition 64. After lapse of the processing time 64a, the second dispersion process is completed.

With reference back to FIG. 14, the controller 30 performs control of a droplet removal process in step S215. The controller 30 controls the container transfer part 220, in a state of gripping the second container 202, so as to transfer the second container 202 from the second dispersion processing part 12 to the liquid removal part 225. The controller 30 controls the liquid removal part 225, thereby supplying an airflow to the outer surface of the second container 202 and removing droplets attached to the outer surface of the second container 202.

In step S216, the controller 30 controls the container transfer part 220 so as to set the second container 202 having been subjected to the droplet removal process, to a holder 201 disposed at the delivery position P9 of the reaction part 200. The controller 30 controls the reaction part 200 so as to intermittently rotate by a predetermined angle every predetermined time.

In step S217, the controller 30 performs control of dispensing a first staining reagent in step S217. The controller 30 controls the reaction part 200 so as to transfer the second container 202 to the first reagent addition position P10. The controller 30 controls the first reagent addition part 21 such that a predetermined amount of the reagent (Rnase) is added to the condensed liquid in the second container 202. The controller 30 controls the reaction part 200 such that the liquid in the second container 202 is heated for a predetermined time at a predetermined temperature, while causing the reaction part 200 to rotate. The predetermined temperature is about 37° C., for example, and the predetermined time is about nine minutes, for example. Accordingly, a process of removing RNA in the cells 51 in the second container 202 is executed. In a predetermined time after the addition of the reagent (Rnase) to completion of the reaction, the second container 202 is moved by the reaction part 200 to the second reagent addition position P11.

In step S218, the controller 30 performs control of dispensing a second staining reagent. The controller 30 controls the second reagent addition part 22 such that a predetermined amount of the reagent (staining liquid) is added to the condensed liquid in the second container 202. The controller 30 controls the reaction part 200 so as to heat the liquid in the second container 202 at the above predetermined temperature, while causing the reaction part 200 to rotate. After the addition of the staining liquid, the second container 202 is moved by the reaction part 200 to the suction position P12 for the sample suction part 230. Before the second container 202 reaches the suction position P12, the reaction (staining) by the staining liquid ends, and the DNA staining process is completed. As a result, a sample 59 for detection by the detection part 120 is prepared.

In step S219, the controller 30 controls the sample suction part 230 so as to suction the prepared sample 59 from the second container 202 moved to the suction position P12. The suctioned sample 59 is supplied from the sample suction part 230 to the flow cell 123 (see FIG. 6) of the detection part 120. Then, the sample preparation process (step S152) is completed.

Step S153 and step S154 in FIG. 13 are executed by the measurement controller 260. When the sample 59 has been supplied to the detection part 120, the measurement controller 260 controls, in step S153, the detection part 120 so as to perform the main measurement on the cells 51 in the sample 59. The measurement controller 260 obtains three pieces of optical information, i.e., forward scattered light waveform data (FS), side scattered light waveform data (SS), and fluorescence waveform data (FL), from the detection part 120. The measurement controller 260 calculates the size of each cell 51, the size of the cell nucleus, and the DNA content (see FIG. 7), from these pieces of optical information.

In step S154, the measurement controller 260 transmits measurement data (the size of the cell 51, the size of the cell nucleus, and the DNA content) to the analysis part 130 via the I/O interface 251.

In step S106, the arithmetic device 131 of the analysis part 130 determines whether or not the measurement data has been received, and repeats the determination until receiving the measurement data. Upon receiving the measurement data, the arithmetic device 131 performs analysis of the measurement data in step S107.

The arithmetic device 131 analyzes the measurement item on the basis of the size of the cell 51, the size of the cell nucleus, and the DNA content. In step S108, the arithmetic device 131 outputs the analysis result to the display part 138.

In step S109, the arithmetic device 131 determines whether or not a shutdown instruction has been received. When not having received the shutdown instruction, the arithmetic device 131 returns the process to step S101. When having received the shutdown instruction, the arithmetic device 131 transmits a shutdown signal to the measurement apparatus 101 in step S110.

The controller 30 of the measurement apparatus 101 determines, in step S155, whether or not the shutdown instruction has been received. When not having received the shutdown instruction, the controller 30 returns the process to step S151. When having received the shutdown instruction, the controller 30 executes, in step S156, a predetermined shutdown process, and shuts down the measurement apparatus 101. In the analysis part 130 as well, the arithmetic device 131 executes a predetermined shutdown process, and shuts down the analysis part 130.

As described above, the sample preparation method according to the present embodiment includes: a step (steps S252, S262) of obtaining a dispersion condition 60 corresponding to the type of the specimen 50; and a step (steps S253, S263) of dispersing aggregated cells 51 contained in the specimen 50, according to the obtained dispersion condition 60. The cell analysis method according to the present embodiment includes: a step (step S152) of preparing the sample 59 by this sample preparation method; a step (steps S217, S218) of staining each cell; a step (step S153) of detecting optical information from the cell in the sample; and a step (step S107) of analyzing a measurement item regarding the proliferative capacity of the cell on the basis of the detected optical information.

Accordingly, cell analysis can be performed by using the sample 59 prepared so as to have an appropriate cell dispersion state, and thus, automation of highly accurate analysis of the cells 51 contained in different types of specimens 50 collected from different organs or sites can be realized. Further, since the cell analysis method according to the present embodiment includes a step of analyzing a measurement item regarding the proliferative capacity of each cell 51 on the basis of detected optical information, an analysis result indicating a morphological change (mutation) of the cell regarding canceration of the cell can be provided on the basis of the optical information of the cell 51 without performing cytology or histology. As a result, with respect to specimens 50 obtained from various tissues or organs, it is possible to provide a useful analysis result that can reduce the burden on laboratory technicians and that can contribute to diagnosis by doctors.

### EXAMPLES

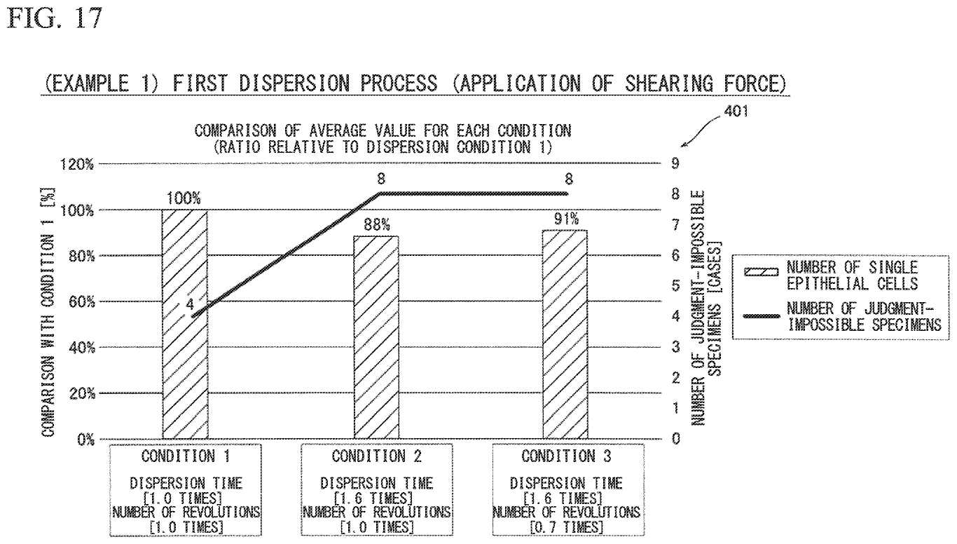

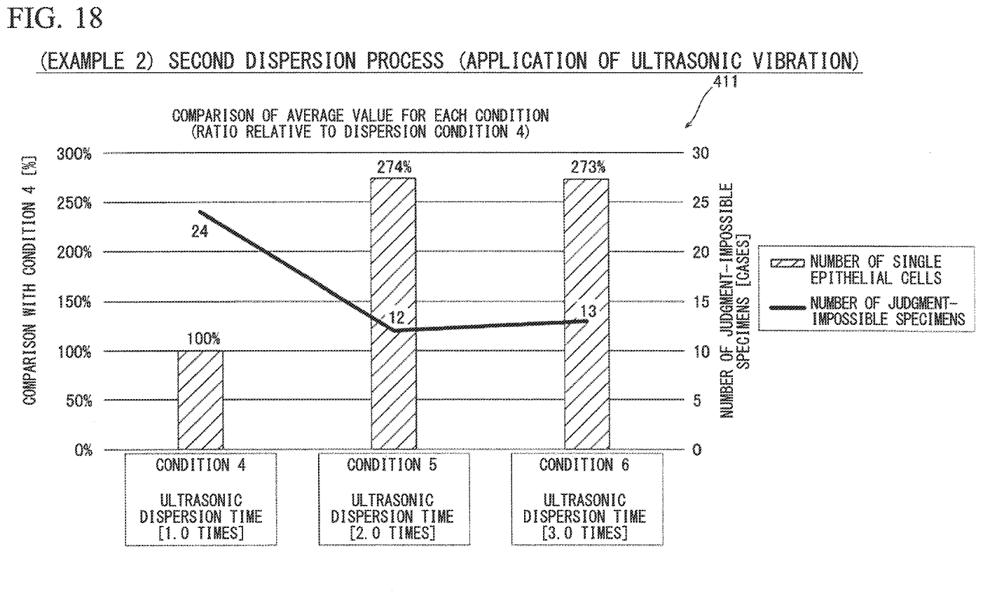

Next, with reference to FIG. 17 and FIG. 18, with respect to the dispersion condition 60 for the dispersion processing part 10, a result obtained by performing the dispersion process under a plurality of different dispersion conditions will be described.

### Example 1

In Example 1, the first dispersion process of applying a shearing force by the first dispersion processing part 11 was performed on the same specimen 50 with the dispersion condition changed, and how the number of cells after the dispersion process changed due to the dispersion condition was examined. As the specimen 50, a specimen obtained by suspending cells collected from the uterine body in a preservation liquid was used.

(Condition 1) The dispersion condition set in condition No. 1 (see FIG. 9) corresponding to epithelial cells of the uterine cervix was used. The dispersion condition of condition No. 1 is a condition that has been optimized for dispersion of epithelial cells of the uterine cervix and with which the best result has been obtained with respect to epithelial cells of the uterine cervix. Hereinafter, condition No. 1 will be used as a reference for the dispersion condition in Example 1. That is, the parameter (dispersion time, the number of revolutions) of the dispersion condition will be represented by a ratio relative to the parameter of condition No. 1.

(Condition 2) Relative to Condition 1, the dispersion time was set to 1.6 times, and the number of revolutions was set to 1.0 times.

(Condition 3) Relative to Condition 1, the dispersion time was set to 1.6 times, and the number of revolutions was set to 0.7 times.

FIG. 17 is a graph 401 showing the number of single cells and the number of judgment-impossible specimens, in Condition 1, Condition 2, and Condition 3. The number of single cells is indicated as a relative value when Condition 1 as the reference is defined as 100% (left vertical axis). The number of single cells is the number of cells dispersed in a single state without being aggregated. The number of judgment-impossible specimens (right vertical axis) indicates the number of specimens in which the detected number of single cells did not reach a statistically significant number of cells set in the cell analyzer 300 and for which judgment (analysis) was determined to be impossible. The number of specimens that were subjected to the experiment was N=21 (specimens). In the graph 401, the number of judgment-impossible specimens out of the 21 specimens is indicated by a line graph.

With reference to the graph 401, in Condition 2 and Condition 3, the number of single cells was decreased when compared with that in Condition 1. Since the number of single cells was decreased, the number of judgment-impossible specimens in Condition 2 and Condition 3 was increased when compared with that in Condition 1.

Therefore, in Example 1, it was found that, in the case of the dispersion process in which a mechanical shearing force is applied by the first dispersion processing part 11, Condition 1 which is optimized for specimens of epithelial cells of the uterine cervix can be applied, as is, also to specimens of epithelial cells of the uterine body, and is appropriate as a dispersion condition. In other words, it was found that a dispersion effect on specimens of epithelial cells of the uterine body cannot be realized by merely increasing the dispersion time of the first dispersion process of applying a shearing force. Therefore, in the example of the dispersion condition 60 shown in FIG. 9, the parameters of the first dispersion process were set to be the same between the first dispersion condition 61 and the second dispersion condition 62.

### Example 2

In Example 2, the second dispersion process of applying ultrasonic vibration on the specimen 50 with the dispersion condition changed was performed in the second dispersion processing part 12, and how the number of cells after the dispersion process changed due to the dispersion condition was examined. As the specimen 50, epithelial cells of the uterine body were used.

In the second dispersion process of Example 2, only the processing time was changed.

(Condition 4) The dispersion condition set to condition No. 1 (see FIG. 9) corresponding to epithelial cells of the uterine cervix was used. The dispersion condition of condition No. 1 has been optimized for dispersion of epithelial cells of the uterine cervix.

(Condition 5) Relative to Condition 4, the processing time was set to 2.0 times.

(Condition 6) Relative to Condition 4, the processing time was set to 3.0 times.

FIG. 18 is a graph 411 showing the number of single cells and the number of judgment-impossible specimens, in Condition 4, Condition 5, and Condition 6. The format of the graph 411 is the same as that of the graph 401 (see FIG. 17) in Example 1 above. The number of specimens that were subjected to the experiment was N=65 (specimens).

With reference to the graph 411, in Condition 5 and Condition 6, the number of single cells was not less than 270% when compared with that in Condition 4. The number of judgment-impossible specimens was reduced to half in Condition 5 (12 specimens) and Condition 6 (13 specimens), when compared with that in Condition 4 (24 specimens).

As described above, in Condition 5 and Condition 6, the number of single cells was significantly increased when compared with that in Condition 4. Therefore, it is understood that, through change of the dispersion condition, a larger number of aggregated cell populations could be dispersed into single cells. In Condition 5 and Condition 6, since the number of single cells was significantly increased, the number of judgment-impossible specimens was greatly decreased when compared with that in Condition 4.

Therefore, in Example 2, it was found that, in the case of the dispersion process in which ultrasonic vibration is applied by the second dispersion processing part 12, the dispersion condition suitable for specimens of epithelial cells of the uterine cervix and the dispersion condition suitable for specimens of epithelial cells of the uterine body are different depending on the type of the specimen. When taking a significant increase in the number of single cells in Conditions 5 and 6 into consideration, it was found that setting the dispersion condition in accordance with the type of the specimen is very effective for increasing the number of single cells and reduction (reduction in variation in the detection result) of the number of judgment-impossible specimens. Therefore, in the example of the dispersion condition 60 shown in FIG. 9, with respect to the second dispersion condition 62 for specimens of the uterine body, parameters corresponding to those in Condition 5 were set in the second dispersion process. The reason for the adoption of Condition 5 is that, while Condition 5 has a dispersion effect equivalent to that of Condition 6, Condition 5 has a shorter processing time and thus has a good processing efficiency. As described above, when the dispersion condition suitable for specimens of epithelial cells of the uterine cervix and the dispersion condition suitable for specimens of epithelial cells of the uterine body are set in accordance with the type of the specimen, an effect of dispersing each of aggregated cells into single cells can be obtained, and cytology of endometrial cancer often observed in late middle-aged women can be automatized as well as cytology of cervical cancer. Cervical cancer and endometrial cancer belong to the same gynecological item, and thus, the fact that cytology of two different types of gynecological items can be performed in automation in the same laboratory contributes to efficiency of tests.

In the Examples, a condition suitable for epithelial cells of the uterine cervix and a condition suitable for epithelial cells of the uterine body were examined. The above result suggests that, also with respect to specimens (oral cavity, esophagus, bronchus, urine, celomic fluid) of other types mentioned above, when a dispersion process is performed under an appropriate dispersion condition corresponding to the type of the specimen, the number of single cells can be increased and variation in the detection result can be reduced.

[Modification]

It should be noted that the embodiment disclosed herein is merely illustrative in all aspects and should not be considered as being restrictive. The scope of the present disclosure is defined not by the description of the above embodiments but by the claims, and further includes meaning equivalent to the claims and all changes (modifications) within the scope.

For example, in FIG. 11, an example in which the dispersion condition 60 is obtained on the basis of the specimen information 90 regarding the specimen 50 has been shown. However, a plurality of methods of obtaining the dispersion condition 60 may be provided.

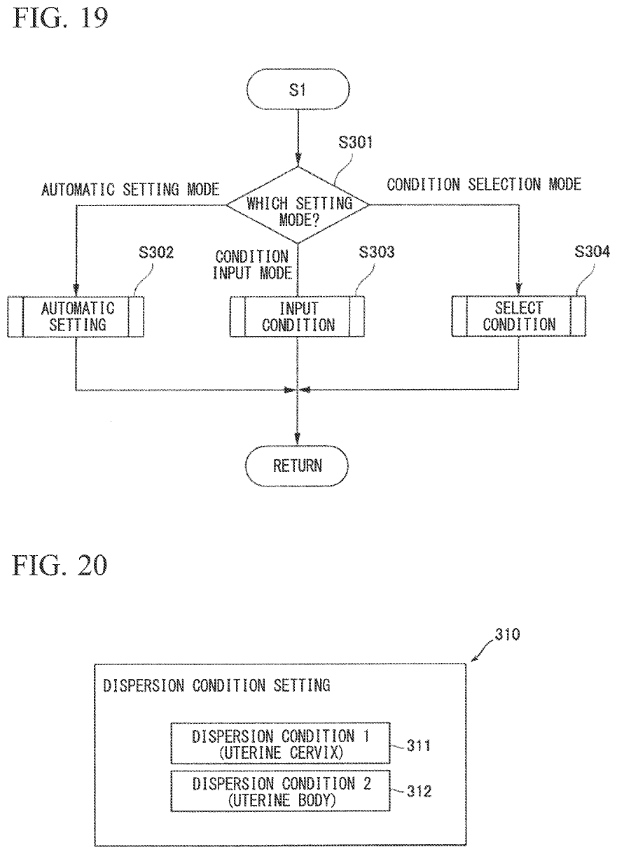

In the example shown in FIG. 19, the step of obtaining the dispersion condition 60 is performed according to one of (1) to (3). (1) Obtaining the dispersion condition 60 on the basis of the specimen information 90 regarding the specimen 50 (step S302). (2) Receiving a selection from among dispersion conditions 60 set in advance for respective types of the specimens 50 (step S304). (3) Receiving an input of the dispersion condition 60 corresponding to the type of the specimen 50 (step S303).

Which of (1) to (3) is used to obtain the dispersion condition 60 is set in advance through mode setting, for example, as shown in FIG. 19. Through step S301 in which the method of obtaining the dispersion condition 60 is selected according to the mode setting, the selected method of obtaining the dispersion condition 60 is executed.

Step S302 of (1) is the obtaining method shown in FIG. 11. According to (1), through obtainment of the specimen information 90 registered with respect to the specimen 50, the dispersion condition 60 suitable for the specimen 50 can be automatically obtained.

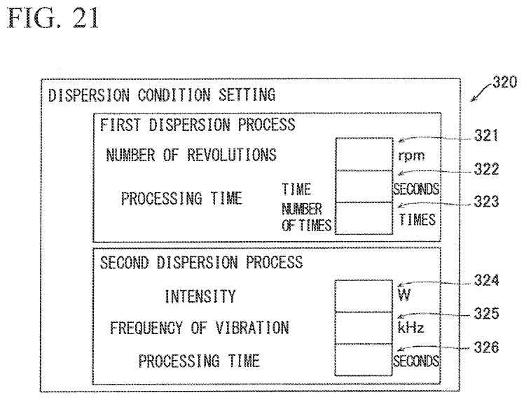

In step S304 of (2), a selection screen 310 (see FIG. 20) for receiving a selection of a dispersion condition 60 set in advance for each type of the specimen 50 is displayed on the display part 138. The selection screen 310 in the example in FIG. 20 includes: a selection button 311 for receiving a selection of a dispersion condition 1 corresponding to a specimen 50 derived from the uterine cervix; and a selection button 312 for receiving selection of a dispersion condition 2 corresponding to a specimen 50 derived from the uterine body. The controller 30 reads out, from the storage 32, the dispersion condition 60 corresponding to the button with which the input has been performed. Accordingly, by the user merely selecting a dispersion condition 60 in accordance with the type of the specimen 50, the dispersion condition 60 suitable for the specimen 50 can be easily obtained. In FIG. 20, for convenience, two types of selections are shown. However, a number of selection buttons corresponding to the number of the types of specimens to be processed can be displayed.

In step S303 of (3), for example, an input screen 320 (see FIG. 21) for receiving an input of a dispersion condition 60 corresponding to the type of the specimen 50 is displayed on the display part 138. The input screen 320 in the example in FIG. 21 includes a number-of-revolutions input field 321, a processing time input field 322, and a number-of-times-of-processing input field 323, as the dispersion condition for the first dispersion process by the first dispersion processing part 11. The input screen 320 includes an intensity input field 324, a frequency-of-vibration input field 325, and a processing time input field 326, as the dispersion condition for the second dispersion process by the second dispersion processing part 12. When the user inputs numerical values corresponding to the type of the specimen 50, to these input fields via the input field, the inputted dispersion condition 60 is transmitted from the analysis part 130 to the controller 30. Accordingly, the user can subjectively set the dispersion condition 60 corresponding to the type of the specimen 50, and thus, the dispersion condition 60 can be optimized.

In the example shown in FIG. 19 to FIG. 21, the controller 30 obtains a dispersion condition 60 through at least one of: the selection screen 310 for receiving a selection of a dispersion condition 60 set in advance for each type of the specimen 50 and displayed on the display part 138; and the input screen 320 for receiving an input of a dispersion condition 60 corresponding to the type of the specimen 50. Accordingly, the user can easily and subjectively set the dispersion condition 60 corresponding to the type of the specimen 50.

Other than the above, although an example in which the dispersion condition 60 is stored in the storage 32 of the controller 30 has been shown, the dispersion condition 60 may be stored in the auxiliary storage device 133 of the analysis part 130, and the controller 30 may obtain the dispersion condition 60 from the analysis part 130.

In FIG. 12, an example in which the type of the specimen is obtained from the host computer 139 by using a specimen ID read by the information reading part 150 has been shown. However, information of the type of the specimen may also be stored in addition to the specimen ID in the information storage medium 151 of the specimen container 141. In this case, the information reading part 150 obtains the type of the specimen as the specimen information 90 from the information storage medium 151, and the controller 30 obtains information of the type of the specimen directly from the information reading part 150.

In FIG. 9, an example in which, with respect to the specimens 50 of epithelial cells of the uterine cervix and the uterine body, two types of the dispersion condition 60 respectively corresponding to the types of the specimens are provided has been shown. However, the present disclosure is not limited thereto. Three or more types of the dispersion condition 60 may be provided. For example, separate dispersion conditions 60 may be respectively set for the specimens 50 of the oral cavity, the esophagus, and the bronchus, other than the uterine cervix and the uterine body. That is, condition No. 3 corresponding to the specimen 50 of the oral cavity, condition No. 4 corresponding to the specimen 50 of the esophagus, and condition No. 5 corresponding to the specimen 50 of the bronchus may be provided in addition to condition No. 1 corresponding to the specimen 50 of the uterine cervix and condition No. 2 corresponding to the specimen 50 of the uterine body. The epithelial cells of the oral cavity, the esophagus, and the bronchus contain a large number of stratified squamous epithelial cells 55. The specimen 50 may be a specimen that contains cells derived from a site other than the uterine cervix, the uterine body, the oral cavity, the esophagus, and the bronchus.

In FIG. 9, an example in which the dispersion condition for the first dispersion process by the first dispersion processing part 11 is common between the first dispersion condition 61 and the second dispersion condition 62 has been shown. However, the present disclosure is not limited thereto. The dispersion condition for the first dispersion process may be different in accordance with the specimen 50. The values of the number of revolutions and the processing time in the first dispersion process are merely examples and are not limited to the numerical values shown in FIG. 9.

In FIG. 9, an example in which, in the second dispersion condition 62, the processing time of the second dispersion process is made longer than that in the first dispersion condition 61, thereby increasing the dispersion effect, has been shown. However, the present disclosure is not limited thereto. For example, the intensity (wattage) of ultrasonic vibration in the second dispersion condition 62 may be set to be higher than that in the first dispersion condition 61. For example, in the first dispersion condition 61, the intensity of ultrasonic vibration is set to 15 W and the processing time is set to five seconds, whereas in the second dispersion condition 62, the intensity of ultrasonic vibration may be set to 20 W and the processing time may be set to five seconds. In this case, the processing time is set to be the same (five seconds) between the first dispersion condition 61 and the second dispersion condition 62. Both of the intensity and the processing time of ultrasonic vibration may be different between the first dispersion condition 61 and the second dispersion condition 62.

In the example shown in FIG. 14 to FIG. 16, a case in which, in step S252, the dispersion condition 63 for the first dispersion process corresponding to the type of the specimen 50 is read out from the storage 32 and obtained, and in step S262, the dispersion condition 64 for the second dispersion process corresponding to the type of the specimen is read out from the storage 32 and obtained, has been shown. However, the present disclosure is not limited thereto. For example, in step S203 in FIG. 14, the dispersion condition 63 for the first dispersion process corresponding to the type of the specimen 50 and the dispersion condition 64 for the second dispersion process corresponding to the type of the specimen may be read out from the storage 32 and obtained.

[1] A sample preparation method for preparing a sample for analyzing cells contained in a specimen, the method comprising:
obtaining a dispersion condition corresponding to a type of the specimen; and
dispersing the cells aggregated and contained in the specimen, according to the obtained dispersion condition.

[2] The sample preparation method according to item [1], wherein
in the obtaining of the dispersion condition, the dispersion condition corresponding to an organ and/or site from which the specimen is derived is obtained.

[3] The sample preparation method according to item [2], wherein
in the obtaining of the dispersion condition, the dispersion condition corresponding to an organ or site collected from one of uterine cervix, uterine body, oral cavity, esophagus, and bronchus, as the type of the specimen, is obtained.

[4] The sample preparation method according to item [2], wherein
in the obtaining of the dispersion condition,
   a first dispersion condition is obtained in a case of the specimen that contains more stratified squamous epithelial cells than simple columnar epithelial cells; and
   a second dispersion condition is obtained in a case of the specimen that contains more simple columnar epithelial cells than stratified squamous epithelial cells, and
the second dispersion condition is a condition that has a dispersion effect higher than that of the first dispersion condition.

[5] The sample preparation method according to item [2], wherein
the dispersing of the cells aggregated and contained in the specimen includes applying ultrasonic vibration to the specimen, and
in the obtaining of the dispersion condition,
a first dispersion condition is obtained when the specimen is a tissue of uterine cervix, and
a second dispersion condition having a processing time for applying the ultrasonic vibration to the specimen longer than that of the first dispersion condition is obtained when the specimen is a tissue of uterine body.

[6] The sample preparation method according item [2], wherein
the obtaining of the dispersion condition includes:
   obtaining, by an information reading part, specimen information regarding the specimen; and
   obtaining, on the basis of the obtained specimen information, the dispersion condition corresponding to the organ and/or site from which the specimen is derived.

[7] The sample preparation method according to item [1], wherein
the dispersing of the cells includes applying a shearing force to the specimen, thereby dispersing the aggregated cells.

[8] The sample preparation method according to item [1], wherein
the dispersing of the cells includes applying ultrasonic vibration to the specimen, thereby dispersing the aggregated cells.

[9] The sample preparation method according to item [1], wherein
the dispersing of the cells includes:
   first dispersion processing of applying a shearing force to the specimen, thereby dispersing the aggregated cells; and
   second dispersion processing of applying ultrasonic vibration to the specimen after the first dispersion processing, thereby dispersing the aggregated cells.

[10] The sample preparation method according to item[9], wherein
in the obtaining of the dispersion condition,
the first dispersion processing has a dispersion condition that is identical between a case where the specimen is a tissue of uterine cervix and a case where the specimen is a tissue of uterine body, and
the second dispersion processing has a dispersion condition that is different between a case where the specimen is a tissue of the uterine cervix and a case where the specimen is a tissue of the uterine body.

[11] The sample preparation method according to item[7], wherein
the dispersion condition in the applying of the shearing force to the specimen includes at least one of a processing time, and a speed of a member that applies the shearing force.

[12] The sample preparation method according to item [8], wherein
the dispersion condition in the applying of the ultrasonic vibration to the specimen includes at least one of a processing time, a frequency of vibration, and an intensity of the ultrasonic vibration.

[13] The sample preparation method according to item [1], further comprising staining the cells.

[14] A cell analysis method for analyzing proliferative capacity of each cell contained in a specimen, the cell analysis method comprising:
preparing a sample by the sample preparation method according to item [1];
detecting optical information from the cell in the sample; and
analyzing a measurement item regarding the proliferative capacity of the cell, on the basis of the detected optical information.

[15] The cell analysis method according to item [14], wherein
the measurement item includes a measurement item to be analyzed on the basis of a size of the cell, a size of a cell nucleus of the cell, and a DNA content of the cell, which are obtained from the optical information detected from the cell in the sample.

[16] A sample preparation apparatus configured to prepare a sample for analyzing cells contained in a specimen, the sample preparation apparatus comprising:
a dispersion processing part configured to disperse the cells aggregated and contained in the specimen; and
a controller programmed to obtain a dispersion condition corresponding to a type of the specimen and control the dispersion processing part so as to perform a dispersion process on the specimen according to the obtained dispersion condition.

[17] The sample preparation apparatus according to item [16], further comprising
an information reading part configured to read specimen information regarding the specimen, wherein
the controller is programmed to obtain the dispersion condition corresponding to an organ and/or site from which the specimen is derived, on the basis of the specimen information read by the information reading part.

[18] The sample preparation apparatus according to item [17], wherein
the controller is programmed to obtain the dispersion condition corresponding to the organ or site collected from one of uterine cervix, uterine body, oral cavity, esophagus, and bronchus, as the type of the specimen, on the basis of the specimen information read by the information reading part.

[19] The sample preparation apparatus according to item [18], wherein
the controller is programmed to
when the specimen is a tissue of the uterine cervix, obtain a first dispersion condition and control the dispersion processing part so as to perform a dispersion process on the specimen according to the obtained first dispersion condition, and
when the specimen is a tissue of the uterine body, obtain a second dispersion condition having a processing time for applying ultrasonic vibration to the specimen longer than that of the first dispersion condition, and control the dispersion processing part so as to perform a dispersion process on the specimen according to the obtained second dispersion condition.

[20] The sample preparation apparatus according to item [16], wherein
the dispersion processing part includes:
   a first dispersion processing part configured to apply a shearing force to the specimen, thereby dispersing the aggregated cells; and
   a second dispersion processing part configured to apply ultrasonic vibration to the specimen, thereby dispersing the aggregated cells, and
the controller is programmed to cause the first dispersion processing part and the second dispersion processing part to operate under the dispersion condition corresponding to the specimen.

[21] The sample preparation apparatus according to item [20], wherein
the controller is programmed to
   control the first dispersion processing part so as to operate under a dispersion condition that is identical between a case where the specimen is a tissue of uterine cervix and a case where the specimen is a tissue of uterine body, and
   control the second dispersion processing part so as to operate under a dispersion condition that is different between a case where the specimen is a tissue of the uterine cervix and a case where the specimen is a tissue of the uterine body.

[22] The sample preparation apparatus according to item [20], wherein
the dispersion condition for the first dispersion processing part includes at least one of a processing time, and a speed of a member that applies the shearing force.

[23] The sample preparation apparatus according to item [20], wherein
the dispersion condition for the second dispersion processing part includes at least one of a processing time, a frequency of vibration, and an intensity of the ultrasonic vibration.

[24] The sample preparation apparatus according to item [21], further comprising:
a staining liquid addition part configured to add, to the specimen, a staining liquid that stains the cells;
a specimen dispensing part configured to dispense the specimen into a container;
a discrimination and replacement part configured to separate the cells from the specimen processed by the first dispersion processing part and subject the separated cells to the second dispersion processing part; and
a reaction part configured to cause the specimen on which a dispersion process has been performed by the second dispersion processing part and the staining liquid added by the staining liquid addition part, to react with each other.

[25] The sample preparation apparatus according to item [24], wherein
the discrimination and replacement part includes a filter for separating the cells.

[26] A cell analyzer configured to analyze proliferative capacity of each cell contained in a specimen, the cell analyzer comprising:
a preparation part implemented as the sample preparation apparatus according to item [16];
a detection part configured to detect optical information from the cell in the sample prepared by the preparation part; and
an analysis part configured to analyze a measurement item regarding the proliferative capacity of the cell, on the basis of the optical information obtained by the detection part.

[27] The cell analyzer according to item [26], wherein
the analysis part analyzes the measurement item on the basis of a size of the cell, a size of a cell nucleus of the cell, and a DNA content of the cell, which are obtained from the optical information detected by the detection part.

What is claimed is:

1. A sample preparation method for preparing a sample for analyzing cells contained in a specimen, the method comprising:
obtaining a dispersion condition corresponding to a type of the specimen; and
dispersing the cells aggregated and contained in the specimen, according to the obtained dispersion condition.

2. The sample preparation method according to claim 1, wherein
in the obtaining of the dispersion condition, the dispersion condition corresponding to an organ and/or site from which the specimen is derived is obtained.

3. The sample preparation method according to claim 2, wherein
in the obtaining of the dispersion condition, the dispersion condition corresponding to an organ or site collected from one of uterine cervix, uterine body, oral cavity, esophagus, and bronchus, as the type of the specimen, is obtained.

4. The sample preparation method according to claim 2, wherein
in the obtaining of the dispersion condition,
a first dispersion condition is obtained in a case of the specimen that contains more stratified squamous epithelial cells than simple columnar epithelial cells; and
a second dispersion condition is obtained in a case of the specimen that contains more simple columnar epithelial cells than stratified squamous epithelial cells, and
the second dispersion condition is a condition that has a dispersion effect higher than that of the first dispersion condition.

5. The sample preparation method according to claim 2, wherein
the dispersing of the cells aggregated and contained in the specimen includes applying ultrasonic vibration to the specimen, and
in the obtaining of the dispersion condition,
a first dispersion condition is obtained when the specimen is a tissue of uterine cervix, and
a second dispersion condition having a processing time for applying the ultrasonic vibration to the specimen longer than that of the first dispersion condition is obtained when the specimen is a tissue of uterine body.

6. The sample preparation method according claim 2, wherein
the obtaining of the dispersion condition includes:
obtaining, by an information reading part, specimen information regarding the specimen; and
obtaining, on the basis of the obtained specimen information, the dispersion condition corresponding to the organ and/or site from which the specimen is derived.

7. The sample preparation method according to claim 1, wherein
the dispersing of the cells includes applying a shearing force to the specimen, thereby dispersing the aggregated cells.

8. The sample preparation method according to claim 1, wherein
the dispersing of the cells includes applying ultrasonic vibration to the specimen, thereby dispersing the aggregated cells.

9. The sample preparation method according to claim 1, wherein
the dispersing of the cells includes:
first dispersion processing of applying a shearing force to the specimen, thereby dispersing the aggregated cells; and
second dispersion processing of applying ultrasonic vibration to the specimen after the first dispersion processing, thereby dispersing the aggregated cells.

10. The sample preparation method according to claim 9, wherein
in the obtaining of the dispersion condition,
the first dispersion processing has a dispersion condition that is identical between a case where the specimen is a tissue of uterine cervix and a case where the specimen is a tissue of uterine body, and
the second dispersion processing has a dispersion condition that is different between a case where the specimen is a tissue of the uterine cervix and a case where the specimen is a tissue of the uterine body.

11. The sample preparation method according to claim 7, wherein
the dispersion condition in the applying of the shearing force to the specimen includes at least one of a processing time, and a speed of a member that applies the shearing force.

12. The sample preparation method according to claim 8, wherein
the dispersion condition in the applying of the ultrasonic vibration to the specimen includes at least one of a processing time, a frequency of vibration, and an intensity of the ultrasonic vibration.

13. The sample preparation method according to claim 1, further comprising staining the cells.

14. A cell analysis method for analyzing proliferative capacity of each cell contained in a specimen, the cell analysis method comprising:
preparing a sample by the sample preparation method according to claim 1;
detecting optical information from the cell in the sample; and
analyzing a measurement item regarding the proliferative capacity of the cell, on the basis of the detected optical information.

15. The cell analysis method according to claim 14, wherein
the measurement item includes a measurement item to be analyzed on the basis of a size of the cell, a size of a cell nucleus of the cell, and a DNA content of the cell, which are obtained from the optical information detected from the cell in the sample.

16. A sample preparation apparatus configured to prepare a sample for analyzing cells contained in a specimen, the sample preparation apparatus comprising:
a dispersion processing part configured to disperse the cells aggregated and contained in the specimen; and
a controller programmed to obtain a dispersion condition corresponding to a type of the specimen and control the dispersion processing part so as to perform a dispersion process on the specimen according to the obtained dispersion condition.

17. The sample preparation apparatus according to claim 16, further comprising
an information reading part configured to read specimen information regarding the specimen, wherein
the controller is programmed to obtain the dispersion condition corresponding to an organ and/or site from which the specimen is derived, on the basis of the specimen information read by the information reading part.

18. The sample preparation apparatus according to claim 17, wherein
the controller is programmed to obtain the dispersion condition corresponding to the organ or site collected from one of uterine cervix, uterine body, oral cavity, esophagus, and bronchus, as the type of the specimen, on the basis of the specimen information read by the information reading part.

19. The sample preparation apparatus according to claim 18, wherein
the controller is programmed to
when the specimen is a tissue of the uterine cervix, obtain a first dispersion condition and control the dispersion processing part so as to perform a dispersion process on the specimen according to the obtained first dispersion condition, and
when the specimen is a tissue of the uterine body, obtain a second dispersion condition having a processing time for applying ultrasonic vibration to the specimen longer than that of the first dispersion condition, and control the dispersion processing part so as to perform a dispersion process on the specimen according to the obtained second dispersion condition.

20. The sample preparation apparatus according to claim 16, wherein
the dispersion processing part includes:
a first dispersion processing part configured to apply a shearing force to the specimen, thereby dispersing the aggregated cells; and
a second dispersion processing part configured to apply ultrasonic vibration to the specimen, thereby dispersing the aggregated cells, and
the controller is programmed to cause the first dispersion processing part and the second dispersion processing part to operate under the dispersion condition corresponding to the specimen.
